(19) United States
(12) Patent Application Publication
(10) Pub. No.: US 2005/0049197 A1
(43) Pub. Date: Mar. 3, 2005

(54) Induction of immune response against desired determinants

(75) Inventors: Alessandro Sette, La Jolla, CA (US); Federico Gaeta, San Rafael, CA (US); Howard M. Grey, La Jolla, CA (US); John Sidney, San Diego, CA (US); Jeffery L. Alexander, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Epimmune Inc., San Diego, CA (US)

(21) Appl. No.: 10/961,789

(22) Filed: Oct. 7, 2004

Related U.S. Application Data
(63) Continuation of application No. 09/707,738, filed on Nov. 6, 2000, now abandoned.
Application No. 09/707,738 is a continuation-in-part of application No. 08/788,822, filed on Jan. 23, 1997, now Pat. No. 6,413,935.
Application No. 09/707,738 is a continuation-in-part of application No. 09/310,462, filed on May 12, 1999, now abandoned.
Application No. 09/310,462 is a continuation-in-part of application No. 08/485,218, filed on Jun. 7, 1995, now abandoned.
Application No. 08/485,218 is a continuation-in-part of application No. 08/305,871, filed on Sep. 14, 1994, now Pat. No. 5,736,142.
Application No. 08/305,871 is a continuation-in-part of application No. 08/121,101, filed on Sep. 14, 1993, now abandoned.
(60) Provisional application No. 60/010,510, filed on Jan. 24, 1996.

(51) Int. Cl. A61K 38/10; A61K 38/08

(52) U.S. Cl. 514/12; 514/13; 514/14; 514/15; 514/16; 514/17

(57) ABSTRACT

This invention provides HLA-DR binding peptides, called “pan DR binding peptides” that are recognized by a broad spectrum of DR molecules. In particular, the present invention provides compositions comprising nucleic acid segments that encode such pan DR binding peptides, which are useful for enhancing the immune response to a desired immunogen.

### CROSS-REFERENCE TO RELATED APPLICATIONS

This application is a continuation of U.S. patent application Ser. No. 09/707,738, filed Nov. 6, 2000, now pending, which is a continuation-in-part of U.S. patent application Ser. No. 08/788,822, filed Jan. 23, 1997, now U.S. Pat. No. 6,413,935, which claims the benefit of U.S. Provisional Application No.60/010,510, filed Jan. 24, 1996, now abandoned. U.S. patent application Ser. No. 09/707,738 is also a continuation-in-part of U.S. patent application Ser. No. 09/310,462, filed May 12, 1999, now abandoned, which is a continuation-in-part of U.S. patent application Ser. No. 08/485,218, filed Jun. 7, 1995, now abandoned, which is a continuation-in-part of U.S. patent application Ser. No. 08/305,871, filed Sept. 14, 1994, now U.S. Pat. No. 5,736,142, which is a continuation-in-part of U.S. patent application Ser. No. 08/121,101, filed Sept. 14, 1993, now abandoned. Each of these applications is incorporated herein by reference in its entirety for all purposes.

### STATEMENT REGARDING FEDERALLY SPONSORED RESEARCH

This invention was made with government support under grants awarded by the National Institutes of Health. The Government has certain rights in the invention.

### BACKGROUND OF THE INVENTION

1. Field of the Invention

This invention pertains to the field of compounds and compositions useful for eliciting and/or enhancing an immune response.

2. Background

MHC molecules are classified as either class I or class II molecules. Class II MHC molecules are expressed by specialized antigen presenting cells (APC) such as macrophages, dendritic cells, or B cells. The class II MHC molecules usually associate with peptide fragments derived from processing of protein antigens which enter an endocytic pathway from the APC exterior. The MHC-peptide complexes are subsequently presented for scrutiny to CD4⁺ T helper cells which can then become activated, proliferate and amplify the immune response to the particular immunogenic peptide that is displayed. Accordingly, activation of T cells requires engagement of the T cell receptor (TCR) by its ligand, namely, a bi-molecular complex of an MHC molecule and a peptide antigen (Shimonkevitz, et al., J. Immunol. 133, 2067-2074 (1984); Babbitt, et al., Nature 317, 359-361 (1985); Buus, et al., Cell 47, 1071-1077 (1986); Townsend, A., and Bodmer, H., Annu. Rev. Immunol. 7, 601-624).

Immunogenic peptides that contain epitopes recognized by T helper cells have been found to be useful in inducing immune responses. The use of helper peptides to enhance antibody responses against particular determinants is described, for instance, in Hervas-Stubbs et al. Vaccine 12:867-871 (1994). The interaction of MHC molecules with T cells, in particular helper T cells, is also involved in determining the nature of the immune response raised against a particular vaccine antigen, which is important to the overall effectiveness of the vaccine.

MHC molecules are also involved in the inappropriate activation of T cells, which is a component of a number of immunopathologies, such as autoimmunity, allograft rejection and allergic responses. Exemplary autoimmune diseases include rheumatoid arthritis, multiple sclerosis, and myasthenia gravis. Allergic responses involving T cell activation include allergies to various pollens, dust mites and the like. In addition, foreign infectious diseases may cause immunopathology (e.g., Lyme disease, hepatitis, LCMV, post-streptococcal endocarditis, or glomerulonephritis). Food hypersensitivities, such as celiac disease and Crohn's disease, as well as other allergic diseases, have been associated with particular MHC molecules or suspected of having an autoimmune component. The most commonly used approach to treating these conditions is to suppress the immune system, typically by using immunosuppressive drugs. Another approach has been proposed for cases in which the MHC molecule associated with the condition is known, involving selective blockade of a given MHC molecule. However, where a number of MHC restrictions are involved, approaches other than selective blockade must be found.

Immunochemical studies of the requirements for peptide binding to class II molecules have been carried out. The binding motifs of several murine and human class II MHC molecules have been defined, and motif analysis by sequencing of naturally processed peptides has also recently been described for various class II types (Rudensky et al., Nature 353, 622-627 (1991); Chicz et al., Nature 358, 764-768 (1992); Hunt et al., Science 256, 1817-1820 (1992); Rudensky et al., Nature 359, 429-431 (1992)). In the case of DR molecules in particular, it has been shown that a large hydrophobic anchor engaging a corresponding hydrophobic pocket of the MHC binding groove is the most crucial determinant of peptide-DR interactions. Several other anchors play definite, albeit less prominent roles and help determine allelic specificity. Recently it has also been emphasized that the peptide backbone of the C-terminal half of the peptide molecule is engaged in direct hydrogen bonding with the walls of the MHC binding groove.

Although allele-specific polymorphic residues that line the peptide binding pockets of MHC molecules tend to endow each molecule with the capacity to bind a unique set of peptides, there are many instances in which a given peptide has been shown to bind to more than one MHC molecule. This has been best documented in the case of the human DR isotype, in which it has been noted that several DR HLA molecules appear to recognize similar peptide motifs. Independently, several investigators reported cross-reactive binding and/or recognition of certain epitopes in the context of multiple DR types, leading to the concept that certain peptides might bind more than one DR molecule (Busch et al., Int. Immunol. 2, 443-451 (1990); Panina-Bordignon et al., Eur. J. Immunol. 19, 2237-2242 (1989); Sinigaglia et al., Nature 336, 778-780 (1988); O'Sullivan et al., J. Immunol. 147, 2663-2669 (1991) Roache et al., J. Immunol. 144, 1849-1856 (1991); Hill et al., J. Immunol. 147, 189-197 (1991)). However, the previously reported epitopes may have the capacity to bind to several DR molecules, but they are by no means universal.

### SUMMARY OF THE INVENTION

The present invention provides methods for a rational approach for selecting or constructing peptides, called “pan DR binding peptides,” that bind to multiple HLA class II HLA-DR molecules; the present invention also provides such pan DR molecules themselves. In preferred embodiments, such pan DR binding peptides serve as potent immunostimulators that can be readily employed in vaccines and other therapeutic agents. In alternative embodiments, the peptides can be used to yield immuno-inhibition. The rational molecular design disclosed here enables the creation or selection of therapeutic and immunostimulating molecules that can be administered across a broad population—for example, up to 50-80% or more of a large fraction of the global population. In contrast, molecules that bind to one HLA DR molecule may only bind to and be immunostimulatory for DR molecules in 10-20% of the same population. Thus, these pan DR binding peptides are highly advantageous for vaccines or therapeutic agents to be used in diverse populations.

The invention thus provides core pan DR binding peptides, and nucleic acids encoding them, as well as derivatives of these peptides that can stimulate an MHC-mediated T cell response. These pan DR binding peptides share a specified pattern of amino acid residues at designated core HLA binding positions. Stimulatory pan DR binding peptides of the invention have, at least some of the non-HLA binding core positions, an amino acid that is relatively large, charged, polar, and/or aromatic. Accordingly, the presence of amino acids at positions that interact with a T cell receptor increases the ability of the peptide to stimulate a T cell response.

In alternative embodiments, the approaches presented here permit the design of peptides that can inhibit an MHC-mediated T cell response. Inhibitory peptides generally include, at the non-HLA binding core residues, relatively small, non-polar, non-charged, non-aromatic amino acids, e.g., alanine. The presence of such amino acids at residues that would otherwise interact with a T cell receptor diminishes the ability of the peptide-MHC complex to interact with the T cell receptors and thereby stimulate T cell activation.

Core pan DR binding peptides (“PADRE®”) are oligopeptides of less than about 50 residues. Preferably, the PADRE® peptide has the formula R₁—R₂—R₃—R₄—R₅, proceeding from the N-terminus to the C-terminus, wherein R₁ consists of at least 2 residues; R₂ is selected from the group consisting of a cyclohexylalanine residue, a tyrosine residue, a phenylalanine residue and conservative substitutions therefor; R₃ is 3 to 5 amino acid residues; R4 is selected from the group consisting of threonine-leucine-lysine, lysine-threonine, and tryptophan-threonine-leucine-lysine, and conservative substitutions therefor; and R5 consists of at least 2 residues.

Certain pan-DR binding (PADRE) peptides have been described in WO 95/07707 and Alexander et al., Immunity 1:751-761 (1994). For example, certain pan DR peptides have the formula R₁—R₂—R₃—R₄—R₅, where R₁ is a D-amino acid followed by alanine or lysine, R₂ is cyclohexylalanine, tyrosine, or phenylalanine, R₃ is 3 or 4 amino acids each of which is selected from the group consisting of alanine, isoleucine, serine and valine; R₄ is threonine-leucine-lysine, lysine-threonine, or tryptophan-threonine-leucine-lysine; and R₅ consists of 2 to 4 amino acids followed by a D-amino acid, where each 2 to 4 amino acids is independently selected from the group consisting of alanine, serine, and valine.

In one group of embodiments, the PADRE® peptide is selected from the group consisting of aAXAAAKTAAAAa, aAXAAAATLKAAa, aAXVAAATLKAAa, aAXIAAATLKAAa, aKXVAAWTLKAAa, and aKFVAAWTLKAAa wherein a is D-alanine, A is L-alanine, X is cyclohexylalanine, K is lysine, T is threonine, L is leucine, V is valine, I is isoleucine, W is tryptophan, and F is phenylalanine. More preferably, the PADRE® peptide is aKXVAAWTLKAAa. In other groups of embodiments, the termini of the peptides can be either in the D- or L-form.

The present invention also provides methods of using the pan DR binding peptides. For example, one can use the stimulatory pan DR peptides to enhance an immune response against an administered immunogen. For example, the pan DR binding peptides can be conjugated with a CTL-inducing peptide or other antigen and administered to induce a CTL response against, e.g., cells that bear a tumor-associated antigen or virally infected cells. In another embodiment, the pan DR peptides are conjugated with antibody-inducing peptides or admixed with an antibody-inducing peptide. Alternatively, one can use the inhibitory pan DR peptides to block an immune response by preventing activation of helper T cells. Due to their cross-reactive class II binding capacity, the inhibitory pan DR binding peptides can be used as therapeutics in the inhibition of T cell mediated events involved in allograft rejection, allergic responses, autoimmunity, and the like.

### DETAILED DESCRIPTION

Definitions

The nomenclature used to describe peptide compounds follows the conventional practice wherein the amino group is presented to the left (the N-terminus) and the carboxyl group to the right (the C-terminus) of each amino acid residue. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G.

The “major histocompatibility complex” or “MHC” contains more than 100 genes in humans. The genes encoding the α and β chains of“MHC class II” molecules are linked within the complex. The particular combination of MHC alleles found on an individual chromosome is known as an “MHC haplotype.” In humans, there exist three pairs of MHC class II α- and β-chain genes. These are referred to as HLA-DR, HLA-DP and HLA-DQ. However, in many haplotypes, the HLA-DR cluster contains an extra β-chain gene whose product can pair with the DRα chain. This means that the three sets of genes actually give rise to four types of MHC class II molecules. All of the class II molecules are capable of presenting antigens to T cells.

An “oligopeptide” or “peptide” as used herein refers to a chain of at least four amino acid or amino acid mimetics. In a preferred embodiment, a peptide of the invention has at least six, more preferably at least nine, and usually fewer than about fifty residues. In a particular embodiment, the peptide has fewer than about twenty-five, and preferably fewer than fifteen, e.g., nine to fourteen residues. The oligopeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

When referring to an amino acid residue in a peptide, oligopeptide or protein the terms “amino acid residue,” “amino acid” and “residue” are used interchangeably and, as used herein, mean an amino acid or amino acid mimetic joined covalently to at least one other amino acid or amino acid mimetic through an amide bond or amide bond mimetic.

As used herein, the term “amino acid,” when unqualified, generally refers to an “L-amino acid” or L-amino acid mimetic, although D-amino acids and L-amino acids may be referred to collectively by the term “amino acid.”

Although the peptides will preferably be substantially free of other naturally occurring proteins and fragments thereof, in some embodiments the peptides can be synthetically conjugated to native fragments or particles.

As used herein, the term “biological activity” means the ability to bind an appropriate MHC molecule and thereby elicit or modulate an immune response. For example, in the case of peptides useful for stimulating immune responses, biological activity comprises inducing a T helper response, which in turn helps induce an immune response against a target antigen or antigen mimetic. In the case of peptides useful for stimulating antibody responses, the peptide will induce a T helper response, which in turn helps induce a humoral response against the target antigen.

A “pan DR binding peptide” or a “PADRE®” peptide (Epimmune Inc., San Diego, Calif.) of the invention is a peptide capable of binding multiple different DR molecules. The pan DR binding peptides can bind to at least 3, more preferably at least 4, 5, or 6 different DR molecules, more preferably 7 or more of the most common DR molecules, more preferably 9 of the most common DR molecules (e.g., DR1, 2w2b, 2w2a, 3, 4w4, 4w14, 4w15, 5, 6, 7, 8, 9, 52a, 52b, 52c, and 53), or alternatively, 50% or more of a panel of DR molecules representative of greater than or equal to 75% of the human population, preferably greater than or equal to 80% of the human population. Preferably the pan DR binding peptides are capable of binding one or more DQ molecules, for example, DQ 3.1.

Throughout this disclosure, results are expressed in terms of IC₅₀'s. Given the conditions in which the assays are run (i.e., limiting MHC and labeled peptide concentrations), these values approximate K_{D} values. It should be noted that IC₅₀ values can change, often dramatically, if the assay conditions are varied, and depending on the particular reagents used (e.g., MHC preparation, etc.). For example, excessive concentrations of MHC will increase the apparent measured IC₅₀ of a given ligand. An alternative way of expressing the binding data, to avoid these uncertainties, is as a relative value to a reference peptide. The reference peptide is included in every assay. As a particular assay becomes more, or less, sensitive, the IC₅₀'s of the peptides tested may change somewhat. However, the binding relative to the reference peptide will not change. For example, in an assay run under conditions such that the IC₅₀ of the reference peptide increases 10-fold, all IC₅₀ values will also shift approximately 10-fold. Therefore, to avoid ambiguities, the assessment of whether a peptide is a good, intermediate, weak, or negative binder should be based on its IC₅₀, relative to the IC₅₀ of the standard peptide.

If the IC₅₀ of the standard peptide measured in a particular assay is different from that reported in Table 1, then it should be understood that the threshold values used to determine good, intermediate, weak, and negative binders should be modified by a corresponding factor.

TABLE 1

| | | | | |
|---|---|---|---|---|
| | | | SEQ | |
| | | | ID | Avg. |
| Allele | Assay standard | Sequence | NO: | IC₅₀ (nM) |

| | | | | |
|---|---|---|---|---|
| DR1 | HA 307-319 | PKYVKQNTLKLAT | 1 | 5 |
| DR2w2b | MBP 78-101 | GRTQDENPVWHFFKNIVTPRTPPP | 2 | 9.1 |
| DR3 | MT 65 kd 3-13 | YKTIAFDEEARR | 3 | 250 |
| DR4w4 | HA 307-319 | PKYVKQNTLKLAT | 1 | 45 |
| DR4w14 | 717.01 combinatorial | YARFQSQTTLKQKT | 4 | 50 |
| DR5 | Tet Tox 830-843 | QYIKANSKFIGITE | 5 | 20 |
| DR7 | Tet Tox 830-843 | QYIKANSKFIGITE | 5 | 25 |
| DR52a | Tet Tox 1272-1284 | NGQIGNDPNRDIL | 6 | 470 |
| DRw53 | 717.01 combinatorial | YARFQSQTTLKQKT | 4 | 58 |
| Dr2w2a | Tet Tox 830-843 | QYIKANSKFIGITE | 5 | 20 |
| DQ3.1 | ROIV | YAHAAHAAHAAHAAHAA | 7 | 15 |
| IAb | ROIV | YAHAAHAAHAAHAAHAA | 7 | 28 |
| IAd | Ova 323-326 | ISQAVHAAHAEINE | 8 | 110 |
| IEd | lambda rep 12-26 | YLEDARRLKAIYEKKK | 9 | 170 |
| IAs | ROIV | YAHAAHAAHAAHAAHAA | 7 | 54 |
| IAk | HEL 46-61 | YNTDGSTDYGILQINSR | 10 | 20 |
| IEk | lambda rep 12-26 | YLEDARRLKAIYEKKK | 9 | 28 |

The PADRE® peptides of the invention, in addition to promoting an immune response against a second determinant, can in certain embodiments serve as target antigens themselves. Thus, for instance, in the case in which the PADRE® peptide is linked to a carbohydrate epitope, the immune response may be to both the PADRE® peptide and the carbohydrate epitope.

As used herein, the term “immunogenic determinant” is any structure that can elicit, facilitate, or produce an immune response, for example carbohydrate epitopes, lipids, proteins, peptides, or combinations thereof. An “antigenic determinant” is a structure that is recognized by one or more products of the immune response (e.g., antibodies, T cell receptors, and the like). The same molecule can be both an antigenic determinant and an immunogenic determinant.

A “CTL epitope” of the present invention is one derived from selected epitopic regions of potential target antigens, such as tumor associated antigens, including, but not limited to, renal cell carcinoma, breast cancer, carcinoembryonic antigens, melanoma antigens such as MAGE, and prostate cancer related antigens; as well as infectious disease/pathogenic antigens such as hepatitis C antigens, Epstein-Barr virus antigens, HIV-1 and HIV-2 antigens, and papilloma virus antigens.

A “humoral response” of the present invention is an antibody-mediated immune response directed towards various regions of an antigenic determinant. One of skill will recognize that a humoral response can also be induced against a PADRE® peptide, wherein the PADRE® peptide would also be included with the determinant; in this embodiment, the elicited immune response can be against both the antibody inducing determinant and the PADRE® peptide.

A “carbohydrate epitope” as used herein refers to a carbohydrate structure, present as a glycoconjugate, e.g., glycoprotein, glycopeptide, glycolipid, and the like, or a polysaccharide, oligosaccharide, or monosaccharide against which an immune response is desired. The carbohydrate epitope may induce a wide range of immune responses. One of skill will recognize that various carbohydrate structures exemplified herein can be variously modified according to standard methods, without adversely affecting antigenicity. For instance, the monosaccharide units of the saccharide may be variously substituted or even replaced with small organic molecules, which serve as mimetics for the monosaccharide.

The phrases “isolated” or “biologically pure” refer to material which is substantially or essentially free from components which normally accompany it as found in its native state. Thus, the peptides of the present invention, when isolated, do not contain materials normally associated with their in situ environment, e.g., MHC molecules on antigen presenting cells. Even where a protein has been isolated to a homogeneous or dominant band, there are trace contaminants in the range of 5-10% of native protein which co-purify with the desired protein. Isolated peptides of this invention do not contain such endogenous co-purified protein.

A “linker” as used herein is any used to provide covalent linkage and spacing between two functional groups (e.g., a PADRE® peptide and a desired determinant). Typically, the linker comprises neutral molecules, such as aliphatic carbon chains, amino acids or amino acid mimetics, which are substantially uncharged under physiological conditions and may have linear or branched side chains. Linkers can also comprise surface-active molecules such as lipids or surfactants. In some cases, the linker may, itself, be immunogenic, although non-therapeutically directed. Various linkers useful in the invention are described in more detail, below. Additionally, the verbs “link” and “conjugate” are used interchangeably herein and refer to covalent attachment of two or more species.

The term “T cell clone” refers to a group of T cells that are progeny of a single virgin lymphocyte and express identical T cell receptor proteins. The term “virgin” lymphocyte is used here as it is used in Stites et al., Basic and Clinical Immunology, 8th Edition, Prentice Hall, Englewood Cliffs, N.J. (1994).

A “T helper peptide” as used herein refers to a peptide recognized by the T cell receptor of T helper cells, generally when the peptide is in combination with an MHC class II molecule. The PADRE® peptides of the present invention are T helper peptides.

### DESCRIPTION OF THE PREFERRED EMBODIMENTS

The present invention provides an approach for rational design of pan DR binding peptides, which peptides can bind multiple MHC DR molecules. In some embodiments, the invention provides peptides that inhibit DR-restricted T cell proliferation, while in other presently preferred embodiments the peptides of the invention act as T helper epitopes that provide help for humoral and/or cell-mediated immune responses. Methods for rational design of such pan DR binding peptides are included.

The pan DR peptides of the invention provide significant advantages over previously available peptides for modulating MHC class II-mediated immune responses. By virtue of their ability to bind to many different DR molecules, the peptides of the invention are useful in modulating T cell responses in a large fraction of the human population. The peptides of the invention can therefore yield enhanced immunogenic or repressive activity, compared to naturally occurring peptides.

Also provided by the invention are methods of using the pan DR peptides to block an immune response by preventing activation of helper T cells. Because of their cross-reactive class II binding capacity, the pan DR binding peptides are used as therapeutics in the inhibition of T cell mediated events involved in allograft rejection, allergic responses, or autoimmunity. Alternatively, the pan DR peptides are useful as an adjuvant-like component in any vaccine formulation. In this embodiment the PADRE® molecules are used to enhance an immune response against an administered immunogen. For instance, the pan DR binding peptides are administered with CTL-inducing peptides to induce a CTL response against, e.g., virally infected cells or cells expressing tumor associated antigens. Alternatively, the pan DR binding peptides are conjugated with a CTL-inducing peptide or carbohydrate and administered to induce a CTL response. In another embodiment, the pan DR peptides are conjugated with antibody-inducing peptides or carbohydrates. In addition, the pan DR binding peptides can be admixed with an antibody-inducing peptide or carbohydrate. The use of helper peptides to enhance antibody responses against particular determinants is described, for instance, in Hervas-Stubbs et al., Vaccine 12:867-871 (1994).

The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2^{nd} Ed., Pierce Chemical Co. (Rockford, Ill., 1984).

Alternatively, recombinant DNA technology can be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Berger and Kimmel, Guide to Molecular Cloning Techniques, Methods in Enzymology 152 Academic Press, Inc., San Diego, Calif. (Berger); Sambrook et al. (1989) Molecular Cloning: A Laboratory Manual (2nd ed.) Vol. 1-3, Cold Spring Harbor Laboratory, Cold Spring Harbor Press, N.Y., (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478 (issued May 21, 1991); and Carr, European Patent No. 0,246,864. Thus, fusion proteins that comprise one or more peptide sequences of the invention can be used to present the appropriate T cell epitope.

As the coding sequence for peptides of the length contemplated herein can be synthesized by chemical techniques, for example, the phosphotriester method of Matteucci et al., J. Am. Chem. Soc. 103: 3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts can also be used, employing suitable vectors and control sequences. It is, of course, appreciated by those of skill in the art that only those embodiments of the invention comprising naturally occurring L-amino acids can be encoded by nucleic acids.

A. Rational Design of Pan DR Peptides

The invention provides pan DR peptides, and methods for the rational design of the pan DR peptides. The methods can be used to select a native sequence or design a pan DR peptide de novo, or to identify modifications that one can introduce into a starting peptide, e.g., one that already binds to one or more DR molecules, in order to broaden the specificity of the starting peptide. Once a pan DR binding core is designed into the peptide, the invention provides methods for rationally designing the remainder of the peptide so that, depending upon the particular modifications introduced, the peptide either inhibits or stimulates T cell-mediated immune responses.

Pan DR Binding Core

The pan DR binding ability of a peptide of the invention is determined by the presence of particular types of amino acids at positions in the peptide that are designated “critical contact sites” (i.e., those residues (or their functional equivalents) that must be present in the peptide so as to confer upon the peptide the ability to bind to one, and in accordance with the invention, preferably several MHC DR molecules). The binding of a peptide to an MHC DR molecule involves the critical interaction between the side chains of these peptide residues with pockets located in the peptide-binding groove of the MHC molecule.

The peptides of the invention that include as the critical contact residues the amino acids as set forth below bind to multiple HLA-DR molecules with at least intermediate affinity, for example. In presently preferred embodiments, the pan DR binding peptides can bind to at least 3, more preferably at least 4, 5, or 6 different DR molecules, more preferably 7 or more of the most common DR molecules, more preferably 9 of the most common DR molecules (e.g., DR1, 2w2b, 2w2a, 3, 4w4, 4w14, 4w15, 5, 6, 7, 8, 9, 52a, 52b, 52c, and 53), or alternatively, 50% or more of a panel of DR molecules representative of greater than or equal to 75% of the human population, preferably greater than or equal to 80% of the human population. The pan DR peptides also preferably bind one or more DQ molecules. Preferably, the peptides bind with high affinity (IC₅₀ of less than about 500 nM). Methods for determining the ability of a peptide to bind to MHC molecules are known to those of skill in the art, and are described in, for example, International Application WO 92/02543.

Using the generic formula Zₙ-X₁X₂X₃X₄X₅X₆X₇X₈X₉-Z_{c} to represent a peptide (see, e.g., Table 4), the “critical contact sites” for pan DR binding are, according to the present invention, at positions X₁, X₂, and X₆. For pan DR binding, X₁ is an amino acid selected from the group consisting of cyclohexylalanine (X), Y, F, M, L, I, V and W, X₂ is an amino acid selected from the group consisting of I and V; and X₆ is an amino acid selected from the group consisting of T, V, M, S, A, C, P, L, and I. “Cyclohexylalanine” includes, but is not limited to, bicyclohexylalanine, dicyclohexylalanine and homocyclohexylalanine. Other molecules that can be used in place of cyclohexylalanine to achieve pan DR binding activity include, for example, 3-cyanophenylalanine, 4-cyanophenylalanine, 3,4-difluorophenylalanine, 3,5-difluorophenylalanine, diphenylalanine, 2-fluorophenylalanine, 4-fluorophenylalanine, homophenylalanine, 4-iodophenylalanine, 4-methylphenylalanine, 2-napthylalanine, 4-nitrophenylalanine, 2-pyridylalanine, 3-pyridylalanine, 1,2,3,4-tetrahydro-3-isoquinolinecarboxylic acid, 4-thiazoylalanine, 3-trifluoromethylphenylalanine, and 4-trifluoromethylphenylalanine; these molecules are also represented by (X) as an amino acid symbol in the tables and peptides herein.

In a presently preferred embodiment, X₁ is cyclohexylalanine, Y, or F, and X₆ is T. In the above formula, X₃, X_{4,} X₅, X₇, X₈, and X₉ are each an amino acid, and Zₙ and Z_{c} each independently comprise 1 to about 20 amino acids (see, Table 3). In some embodiments, Zₙ and Z_{c} each comprise about 20 amino acids or less, 15 amino acids or less, about 10 amino acids or less, or about 5 amino acids or less. Zₙ and Z_{c}, in some embodiments, are at least 2 amino acids in length.

In some embodiments (see, e.g., Table 4), the pan DR peptides of the invention are described using the formula Zₙ-X₀X₁X₂X₃X₄X₅X₆X₇X₈X₉-Z_{c}, wherein: X₀ is an amino acid selected from the group consisting of K and A; X₁ is an amino acid selected from the group consisting of (X), Y and F; X₂ is an amino acid selected from the group consisting of I and V; and X₆ is T.X₃, X₄, X₅, X₇, X₈, and X₉ are each an amino acid, and Zₙ and Z_{c} each comprise 1 to 20 amino acids (see, Table 4). In some embodiments, Zₙ and Z_{c} each comprise about 20 amino acids or less, 15 amino acids or less, about 10 amino acids or less, or about 5 amino acids or less. Zₙ and Z_{c}, in some embodiments, are at least 2 amino acids in length.

The identity of amino acids X₀, X₃, X₄, X₅, X₇, X₈, and X₉, and also Zₙ and Z_{c} positions can determine whether a peptide will inhibit or stimulate T cell mediated immune responses, as described below. Where the amino acid present at a particular position is not correlated to a particular biological activity desired for the peptide, these positions can be any amino acid. In any case, however, it is desirable to use at each position an amino acid that does not interfere with the pan DR binding ability of the peptide.

In some embodiments, the peptides of the invention have, at their amino- and/or carboxy- termini, one or more amino acids, or mimetics thereof, that are chosen such as to alter physical or chemical properties of the peptide. These residues, designated Zₙ and Z_{c}, in the formulas set forth herein, can be chosen so as to affect properties such as binding, stability, bioavailability, ease of linking, and the like. Zₙ and Z_{c} are each typically between about zero and about 20 amino acids (or mimetics) in length, more preferably between about one and about 15 amino acids, or zero to about 10 amino acids in length. Generally, the total length of the pan DR peptides of the invention is between about nine and about 50 amino acids. More preferably, the pan DR peptides are between about nine and about 40 amino acids, and still more preferably are between about 12 and about 25 amino acids in length.

To identify degenerate DR-binding epitopes, peptides can be screened for binding to sequential panels of DR molecules. For example, peptides that are capable of binding multiple DR molecules can be identified by the use of combined DR1-4-7 motifs. The composition of suitable panels, and the phenotypic frequency of associated antigens, are shown in Table 2. (See, Imanishi, T. et al., Allele and haplotype sequences for HLA and complement loci in various ethnic groups. In Proceedings of the Eleventh International Histocompatibility Workshop and Conference, Vol. 1, K. Tsuji, M. Aizaqa, and T. Sasazuki, et al. (Tokyo, Japan, Oxford University Press).) For example, all peptides can be initially tested for binding to the molecules in the primary panel (e.g., DR1, DR4w4 and DR7 (see, e.g., Southwood et al. (1998) J. Immunol. 160: 3363-3373). Next, whether the peptides that exhibit degenerate binding behavior for the primary panel would also bind other common DR types as well is examined. Only peptides that bind (for screening, a peptide is considered a “binder” if it has an IC₅₀ of less than or equal to 1000 nM) to at least two of these three molecules are then tested for binding in the secondary assays (e.g., DR2w2β1, DR2w2β2, DR6w19 and DR9). Finally, only peptides that bind to at least two of the four secondary panel molecules, and thus four of seven molecules in total, are screened for binding in the tertiary assays (e.g., DR4w15, DR5w11 and DR8w2). If desired, peptides that bind to members of the tertiary panel can be tested for ability to bind to a quaternary panel (e.g., DR3, DR6, DR8, DR9, and DR12).

TABLE 2
HLA-DR Screening Panels

| Screening | | | Representative Assay | | Phenotypic Frequencies | | | | | |
|---|---|---|---|---|---|---|---|---|---|---|
| Panel | Antigen | Alleles | Allele | Alias | Cauc. | Blk. | Jpn. | Chn. | Hisp. | Avg. |
| Primary | DR1 | DRB1*0101-03 | DRB1*0101 | (DR1) | 18.5 | 8.4 | 10.7 | 4.5 | 10.1 | 10.4 |
| | DR4 | DRB1*0401-12 | DRB1*0401 | (DR4w4) | 23.6 | 6.1 | 40.4 | 21.9 | 29.8 | 24.4 |
| | DR7 | DRB1*0701-02 | DRB1*0701-02 | (DR7) | 22.6 | 11.1 | 1.0 | 15.0 | 16.6 | 14.0 |
| | Panel Total | | | | 59.6 | 24.5 | 49.3 | 38.7 | 51.1 | 44.6 |
| Secondary | DR2 | DRB1*1501-03 | DRB1*1501 | (DR2w2β1) | 19.9 | 14.8 | 30.9 | 22.0 | 15.0 | 20.5 |
| | DR2 | DRB5*0101 | DRB5*0101 | (DR2w2β2) | — | — | — | — | — | — |
| | DR9 | DRB1*09011, 09012 | DRB1*0901 | (DR9) | 3.6 | 4.7 | 24.5 | 19.9 | 6.7 | 11.9 |
| | DR13 | DRB1*1301-06 | DRB1*1302 | (DR6w19) | 21.7 | 16.5 | 14.6 | 12.2 | 10.5 | 15.1 |
| | Panel Total | | | | 42.0 | 33.9 | 61.0 | 48.9 | 30.5 | 43.2 |
| Tertiary | DR4 | DRB1*0405 | DRB1*0405 | (DR4w15) | — | — | — | — | — | — |
| | DR8 | DRB1*0801-5 | DRB1*0802 | (DR8w2) | 5.5 | 10.9 | 25.0 | 10.7 | 23.3 | 15.1 |
| | DR11 | DRB1*1101-05 | DRB1*1101 | (DR5w11) | 17.0 | 18.0 | 4.9 | 19.4 | 18.1 | 15.5 |
| | Panel Total | | | | 22.0 | 27.8 | 29.2 | 29.0 | 39.0 | 29.4 |
| Quaternary | DR3 | DRB1*0301-2 | DRB1*0301 | (DR3w17) | 17.7 | 19.5 | 0.4 | 7.3 | 14.4 | 11.9 |
| | DR12 | DRB1*1201-02 | DRB1*1201 | (DR5w12) | 2.8 | 5.5 | 13.1 | 17.6 | 5.7 | 8.9 |
| | Panel Total | | | | 20.2 | 24.4 | 13.5 | 24.2 | 19.7 | 20.4 |

Modifications of peptides with various amino acid mimetics or D-amino acids, for instance at the N— or C-termini, are useful for instance, in increasing the stability of the peptide in vivo. Such peptides can be synthesized as “inverso” or “retro-inverso” forms, that is, by replacing L-amino acids of a sequence with D-amino acids, or by reversing the sequence of the amino acids and replacing the L-amino acids with D-amino acids. As the D-peptides are substantially more resistant to peptidases, and therefore are more stable in serum and tissues compared to their L-peptide counterparts, the stability of D-peptides under physiological conditions may more than compensate for a difference in affinity compared to the corresponding L-peptide. Further, L-amino acid-containing peptides with or without substitutions can be capped with a D-amino acid to inhibit exopeptidase destruction of the antigenic peptide. Accordingly, stability can be increased by introducing D-amino acid residues at the C and N termini of the peptide. Previous studies have indicated that the half-life of L-amino acid-containing peptides in vivo and in vitro, when incubated in serum-containing medium, can be extended considerably by rendering the peptides resistant to exopeptidase activity by introducing D-amino acids at the C and N termini. Other methods, e.g., amidation of the carboxy termini of the peptides, can also impart increased stability.

Stability can be assayed in a number of ways. For instance, peptidases and various biological media, such as human plasma and serum, have been used to test stability. See, e.g., Verhoef et al., Eur. J. Drug Metab. Pharmacokin. 11, 291-302 (1986); Walter et al., Proc. Soc. Exp. Biol. Med. 148, 98-103 (1975); Witter et al., Neuroendocrinology 30, 377-381 (1980); Verhoef et al., J. Endocrinology 110, 557-562 (1986); Handa et al., Eur. J. Pharmacol. 70, 531-540 (1981); Bizzozero et al., Eur. J. Biochem. 122, 251-258 (1982); Chang, Eur. J. Biochem. 151, 217-224 (1985).

The peptides or analogs of the invention can be modified by altering the order or composition of certain residues, it being readily appreciated that certain amino acid residues essential for biological activity, e.g., those at critical contact sites, may generally not be altered without an adverse effect on biological activity. The non-critical amino acids need not be limited to those naturally occurring in proteins, such as L-α-amino acids, or their D-isomers, but may include non-protein (including artificial) amino acids as well, such as β-γ-δ-amino acids, as well as many derivatives of L-α-amino acids. Accordingly, a peptide of the present invention can generally comprise either L-amino acids or D-amino acids, but not D-amino acids or non-protein (e.g., artificial) amino acids within a core binding region. If a peptide has no D-amino acids or other stabilizing modifications, it is sometimes desirable to increase the length of the peptide to provide for increased stability. Thus, it is generally preferred that if only L-amino acids are included in a peptide, or are encoded by a nucleic acid, that the peptide have several additional amino acids in the Zₙ and Z_{c} regions to adjust for potential degradation.

T Cell Inhibitory Peptides

The invention also provides peptides, and methods for the rational design of such peptides, that can inhibit an antigen-specific T cell-mediated immune response in vivo. These peptides are based on a pan DR binding core as described above. Once one has designed a pan DR peptide by introducing the specified amino acids at the “critical contact residues” for MHC binding, the peptide can be further designed so as to inhibit an immune response. The rational design of such inhibitory peptides involves the use of a non-polar, non-charged, non-aromatic, non-bulky amino acid at peptide positions other than those that are “critical contact residues.” These residues are positioned at sites in the molecules that are capable of being involved in the interaction between the peptide when complexed to MHC and a T cell receptor. The interactions between a peptide and a receptor generally involve the side chains of amino acids having a charge, hydrophobicity, or some other feature for which the receptor has a corresponding moiety that is able to interact with the side chain (e.g., a charge opposite that of the side chain on the peptide). Accordingly, by using an amino acid that is identified or engineered so as to possess a relatively small, inconspicuous side chain(s), the ability of the peptide to interact with the T cell receptor is reduced or eliminated.

Again using the generic formula Zₙ-X₁X₂X₃X₄X₅X₆X₇X₈X₉-Z_{c} to represent a peptide, a T cell inhibitory peptide will have a binding core of critical contact residues as set forth above, and one or more of the residues that are not “critical contact residues”, i.e., a non-HLA binding pocket residues X₃, X₄, X₅, X₇, X₈, and X₉ will be a non-polar, non-charged, non-aromatic, non-bulky amino acid. Most preferably, all six of these residues are a non-polar, non-charged, non-aromatic, non-bulky amino acid (Table 3). Generally, at least one of these residues, more preferably at least two, still more preferably at least three, four, or five residues is a non-polar, non-charged, non-aromatic, non-bulky amino acid. X₅ in particular, either alone or in combination with other residues, is a non-polar, non-charged, non-aromatic, non-bulky amino acid (e.g., A) in some preferred embodiments.

The non-polar, non-charged, non-aromatic, non-bulky amino acids used at the specified positions include, for example, alanine and other amino acids with these characteristics. Properties of amino acids, e.g., charge, polarity, etc., are described in, for example, Creighton, ed. Proteins: structures and molecular properties, 2^{nd} Ed. (Freeman & Co., New York, 1993); Page, David S., Principles of biological chemistry, 2^{nd} Ed., (Willard Grant Press, Boston, 1981); and Stryer, Lubert, Biochemistry, 3^{rd} Ed. (Freeman & Co., San Francisco, 1988). In a presently preferred embodiment, an alanine is used at these “non-critical” positions, which are associated with T cell receptor, rather than HLA pocket binding properties.

An increase in inhibitory activity can be achieved by including one or more such amino acids in Zₙ and/or Z_{c}, particularly the amino acid residue that is immediately adjacent to the binding core. For example, in a preferred embodiment at least one of, and preferably both, of Zₙ and Z_{c} is an alanine or other non-polar, non-charged, non-aromatic, non-bulky amino acid.

In some embodiments, the pan DR peptides of the invention are described using the formula Zₙ-X₀X₁X₂X₃X₄X₅X₆X₇X₈X₉-Z_{c}. The peptides include, for pan DR binding ability, X₀, which is an amino acid selected from the group consisting of K and A; X₁, which is an amino acid selected from the group consisting of (X), Y and F; X₂, which is an amino acid selected from the group consisting of I and V; and X₆, which is T (see, Table 4). To obtain or design an inhibitory peptide, at least one and more preferably more of residues X₃, X_{4,} X₅, X₇, X₈, and X₉ are non-polar, non-charged, non-aromatic, non-bulky amino acids. In some preferred embodiments, at least X₅ is alanine, glycine, proline, or similar residues; alanine is a particularly preferred residue. For inhibitory activity, X₀ preferably is also a non-polar, non-charged, non-aromatic, non-bulky amino acid, in particular alanine.

Once an inhibitory peptide is designed using the methods of the invention, it is desirable to test the peptide to ensure that it exhibits pan DR binding ability and, if desired, DQ binding ability. Those peptides that do exhibit such binding ability are then tested for biological activity. The biological activity of the peptides obtained using the rational design methods described above can be assayed in a variety of systems. Typically, the ability to inhibit antigen-specific T cell activation is tested. In one exemplary protocol, an excess of peptide is incubated with an antigen-presenting cell of known MHC expression, (e.g., DR1) and a T cell clone of known antigen specificity (e.g., tetanus toxin 830-843) and MHC restriction (again, DR1), and the antigenic peptide itself (i.e., tetanus toxin 830-843). The assay culture is incubated for a sufficient time for T cell proliferation, such as four days, and proliferation is then measured using standard procedures, such as pulsing with tritiated thymidine during the last 18 hours of incubation. The percent inhibition, compared to the controls which received no inhibitory peptide, is then calculated.

The capacity of peptides to inhibit antigen presentation in an in vitro assay has been correlated to the capacity of the peptide to inhibit an immune response in vivo. In vivo activity can be determined in animal models, for example, by administering an antigen known to be restricted to the particular MHC molecule recognized by the peptide, along with the new immunomodulatory peptide. T lymphocytes are subsequently removed from the animal and cultured with a dose range of antigen. Inhibition by the engineered peptide of stimulation by the control peptide is measured by conventional means, e.g;, pulsing with [³H]-thymidine, and comparing to appropriate controls. Certain experimental details will of course be apparent to the skilled artisan. See also, Adorini et al., Nature 334, 623-625 (1988).

A large number of cells with defined MHC molecules, particularly MHC class II molecules, are known and readily available from, for instance, the American Type Culture Collection (see, e.g., online catalog at www.atcc.org; “Catalogue of Cell Lines and Hybridomas,” 8th edition (1994) Manassas, Va.).

Examples of pan DR binding peptides of the invention that can inhibit a T cell mediated immune response include, for example:
aA(X)VAAATLKAAa
aA(X)IAAATLKAAa.

T Cell Stimulatory Peptides

In other particularly preferred embodiments, the invention provides peptides, and methods for rational design of such peptides, that can act as helper epitopes for the induction of humoral and/or T cell mediated immune responses. The principle which underlies the design of T cell stimulatory peptides is the converse of that for T cell inhibitory peptides. Whereas the design of inhibitory peptides involves the replacement of amino acid side chains that have significant binding energy with a T cell receptor with amino acids that do not, a T cell stimulatory peptide will have at these T cell receptor binding positions more bulky, hydrophobic, or charged amino acids. The presence of such residues can increase the affinity of interaction between the peptides and T cell receptors, thus increasing the immunogenicity of the peptides when inducing T cell mediated responses.

The T cell stimulatory peptides of the invention have, again using the generic formula Zₙ-X₁X₂X₃X₄X₅X₆X₇X₈X₉-Z_{c}, a “binding core” as set forth above (see, e.g., Table 4). The “critical contact sites” for pan DR binding are, according to the present invention, at positions X₁, X₂, and X₆. For pan DR binding, X₁ is an amino acid selected from the group consisting of cyclohexylalanine (X), Y, F, M, L, I, V and W, X₂ is an amino acid selected from the group consisting of I and V; and X₆ is an amino acid selected from the group consisting of T, V, M, S, A, C, P, L, and I. In the T cell stimulatory peptides, one or more of the non-HLA pocket residues X₃, X₄, X₅, X₇, X₈, and X₉ will be an amino acid that has a side chain having significant binding energy for a T cell receptor. For example, one or more of these amino acids will be polar, charged, aromatic, and/or bulky. Preferably, at least one, more preferably at least two, still more preferably at least three, four, or five, and most preferably all six of these residues are a polar, charged, aromatic, and/or bulky amino acid. In most preferred embodiments, each of X₃, X₄, X₅, X₇, X₈, and X₉ is a polar, charged, aromatic, and/or bulky amino acid. In preferred embodiments, at least X₅ is such an amino acid, either alone or in combination with other amino acids. Moreover, an increase in stimulatory activity can be achieved by including one or more such amino acids in Zₙ and/or Z_{c}, particularly the amino acid residue that is immediately adjacent to the binding core. See Table 3 for a listing of amino acids that are preferred at the particular positions.

In some embodiments, the pan DR stimulatory peptides of the invention are described using the formula Zₙ-X₀X₁X₂X₃X₄X₅X₆X₇X₈X₉-Z_{c}. The residues involved in pan DR binding ability are as described above; namely, X₀ is an amino acid selected from the group consisting of K and A; X₁ is an amino acid selected from the group consisting of (X), Y and F; X₂ is an amino acid selected from the group consisting of I and V; and X₆ is T. To obtain an stimulatory peptide, at least one of residues X₃, X₄, X₅, X₇, X₈, and X₉ is a polar, charged, aromatic, and/or bulky amino acid. In presently preferred embodiments, X₀ is also a polar, charged, aromatic, and/or bulky amino acid. See Table 4 for a listing of amino acids that are preferred at the particular positions. The non-polar, non-charged, non-aromatic, non-bulky amino acids used at the specified positions include, for example, tryptophan or lysine. In a presently preferred embodiment, a tryptophan is used at these positions. In additional preferred embodiments, X₇ or X₈, or both, are K.

Once a T cell stimulatory peptide is designed using the methods of the invention, it is desirable to test the peptide to ensure that it exhibits pan DR binding ability. Those peptides that do exhibit such binding ability are then tested for biological activity. For example, the peptides can be tested for ability to induce T cell activation in an in vitro assay system. Induction of T cell activation is a reliable indicator of a peptide's ability to induce T cell helper capacity. Suitable in vitro assays for T cell activation, e.g., T cell proliferation and lymphokine secretion assays, are known to those of skill in the art.

TABLE 3

| | N-terminal (Zₙ) | 1 | 2 | 3 | 4 | 5 | 6 | 7 | 8 | 9 | C-terminal (Z_{c}) |
|---|---|---|---|---|---|---|---|---|---|---|---|
| pan-DR | Pref. at least 1 amino | (X), | I, V | Any | Any | Any | T, V, M, | Any | Any | Any | Pref. at least 1 amino |
| Binding Core | acid for stability; | Y, F, | | | | | S, A, C, | | | | acid for stability; |
| | preferably at least | M, L, | | | | | P, L, or I | | | | preferably at least one |
| | one D amino acid | I, V or W | | | | | | | | | D amino acid |
| Inhibitory- | A¹ | | | A¹ | A¹ | A¹ | | A¹ | A¹ | A¹ | A¹ |
| same as core | | | | | | | | | | | |
| plus: | | | | | | | | | | | |
| Stimulatory- | W² | | | W² | W² | W² | | W² | W² | W² | W² |
| same as core | | | | | | | | | | | |
| plus: | | | | | | | | | | | |

TABLE 4

| | N-terminal (Zₙ) | 0 | 1 | 2 | 3 | 4 | 5 | 6 | 7 | 8 | 9 | C-terminal (Z_{c}) |
|---|---|---|---|---|---|---|---|---|---|---|---|---|
| pan-DR | Pref. at least 1 | K, A | (X), Y, F | I, V | Any | Any | Any | T | Any | Any | Any | Pref. at least 1 amino |
| Binding Core | amino acid for | | | | | | | | | | | acid for stability; |
| | stability; | | | | | | | | | | | preferably at least one |
| | preferably at least | | | | | | | | | | | D amino acid |
| | one D amino acid | | | | | | | | | | | |
| Inhibitory- | A¹ | A¹ | | | A¹ | A¹ | A¹ | | A¹ | A¹ | A¹ | A¹ |
| same as core | | | | | | | | | | | | |
| plus: | | | | | | | | | | | | |
| Stimulatory- | W² | W² | | | W² | W² | W² | | W² | W² | W² | W² |
| same as core | | | | | | | | | (esp. | (esp. | | |
| plus: | | | | | | | | | K) | K) | | |

¹Or other non-polar, non-charged, non-bulky, non-aromatic amino acid
²Or other polar, charged, bulky, or aromatic amino acid

Examples of pan DR binding peptides of the invention that can induce. or enhance a T-helper cell mediated immune response include, for example, the first 8 peptides listed in Table 9. This Table provides an illustration of various substitutions that one can make to obtain different pan DR stimulatory peptides. For example, the peptide 965.10 is a synthetic peptide, having a non-naturally occurring cyclohexylalanine or similar peptide at position X₂ and being flanked on each end by D-amino acids. An analogous preferred peptide has a substitution, e.g., phenylalanine, at position X₂ of peptide 965.10. To obtain an all-natural yet analogous peptide, the D-amino acids at each end can be replaced by L-amino acids in addition to the substitution of a naturally occurring amino acid for the cyclohexylalanine; an all-L-amino acid peptide such as this can be prepared and/or administered using nucleic acids that encode the peptide. Each of these three peptides can then be subjected to an additional substitution at position X₆, as illustrated in Table 5. For example, the tryptophan at position X₆ of peptide 965.10 or its two derivatives can be replaced by asparagine, tyrosine, lysine, histidine, or alanine without loss of stimulatory activity. Thus, preferred peptides include those shown in Table 5.

TABLE 5

| Amino acid at | | Replacement of | All-Natural (no D-amino acids |
|---|---|---|---|
| Position X₆ | Synthetic | Cyclohexylalanine | or cyclohexylalanine) |
| W | aK(X)VAAWTLKAAa | aKFVAAWTLKAAa | AKFVAAWTLKAAA (SEQ ID |
| | (SEQ ID NO: 11) | (SEQ ID NO: 12) | NO: 13) |
| N | aK(X)VAANTLKAAa | aKFVAANTLKAAa | AKFVAANTLKAAA (SEQ ID |
| | (SEQ ID NO: 14) | (SEQ ID NO: 15) | NO: 16) |
| Y | aK(X)VAAYTLKAAa | aKFVAAYTLKAAa | AKFVAAYTLKAAA |
| | (SEQ ID NO: 17) | (SEQ ID NO: 18) | (SEQ ID NO: 19) |
| K | aK(X)VAAKTLKAAa | aKFVAAKTLKAAa | AKFVAAKTLKAAA (SEQ ID |
| | (SEQ ID NO: 20) | (SEQ ID NO: 21) | NO: 22) |
| H | aK(X)VAAHTLKAAa | aKFVAAHTLKAAa | AKFVAAHTLKAAA |
| | (SEQ ID NO: 23) | (SEQ ID NO: 24) | (SEQ ID NO: 25) |
| A | aK(X)VAAATLKAAa | aKFVAAATLKAAa | AKFVAAATLKAAA (SEQ ID |
| | (SEQ ID NO: 26) | (SEQ ID NO: 27) | NO: 28) |

B. Computer Screening of Protein Sequences for Pan DR Binding Peptides

Computer programs that allow the rapid screening of protein sequences for the occurrence of the subject pan DR binding motifs are encompassed by the present invention. These programs are implemented to analyze any identified amino acid sequence, or to operate on an unknown sequence and simultaneously determine the sequence and identify motif-bearing epitopes thereof. Generally, the identified sequences will be from a pathogenic organism or a tumor-associated peptide.

In utilizing computer screening to identify peptide epitopes, a protein sequence or translated sequence may be analyzed using software developed to search for motifs, for example the “FINDPATTERNS” program (Devereux, et al. Nucl. Acids Res. 12:387-395, 1984) or MotifSearch 1.4 text string software program (D. Brown, San Diego, Calif.) to identify potential peptide sequences containing a pan DR binding motif.

As noted above, the pan DR motif includes the presence of particular amino acid residues, as set forth in Tables 3 and 4, at core positions X₁, X₂, and X₆. In certain embodiments, the motif may be further refined to identify those peptides that bear stimulatory or inhibitory residues, as defined in Tables 3 and 4, at particular positions of the peptide epitope.

As appreciated by one of ordinary skill in the art, a large array of computer programming software and hardware options are available in the relevant art which can be employed to implement the motifs of the invention in order to evaluate (e.g., without limitation, to identify epitopes, identify epitope concentration per peptide length, or to generate analogs) known or unknown peptide sequences. Furthermore, such calculations can be made mentally.

Once peptide sequences that bear pan-DR binding motifs have been identified, the corresponding peptides can be synthesized and tested to confirm the ability to bind multiple DR molecules. Those peptides that exhibit pan DR binding activity, i.e., peptides that bind to multiple, preferably at least three, four, five or six allele-specific DR molecules, may then be tested for the ability to stimulate HTL activity using in vitro assays, e.g. T cell proliferation (see, e.g. Alexander et al., J. Immunol. 159:4753-4761, 1997) known by those in the art.

Pan DR motif-bearing peptides identified as described above may further be modified to exhibit enhanced T cell stimulating activity or inhibitory T cell activity. Such modifications may be made using the inhibitory or stimulatory residues identified in Tables 3 and 4 as guidance. The modified peptides may then be tested for increased T cell stimulating or inhibitory activity.

C. Use of Pan DR Peptides to Modulate Immune Responses

The invention also provides methods and compositions in which pan DR peptides are used for modulating immune responses. The particular peptide employed determines whether the modulation is inhibition or stimulation.

Inhibition of T Cell-Mediated Immune Responses

In one embodiment, the present invention provides methods of using the inhibitory pan DR peptides to block an immune response by preventing activation of helper T cells. As a result of their cross-reactive class II binding capacity, the pan DR binding peptides can be used as therapeutics in the inhibition of T cell mediated events involved in allograft rejection, allergic responses, autoimmunity, and the like. The administration of the inhibitory pan DR peptides of the invention to an animal in need of treatment for such conditions and subsequent binding of the peptide to the MHC molecules can block the ability of the MHC to bind peptides that are involved in eliciting or exacerbating the inappropriate immune reaction.

The pan DR binding peptides can be used to treat a variety of conditions involving unwanted T cell reactivity. Examples of diseases which can be treated using pan DR binding peptides include autoimmune diseases (e.g., rheumatoid arthritis, multiple sclerosis, and myasthenia gravis), allograft rejection, allergies (e.g., pollen or pet allergies), Lyme disease, hepatitis, LCMV, post-streptococcal endocarditis, or glomerulonephritis, and food hypersensitivities.

Stimulation of Helper T Cell Mediated Immune Responses

Particularly preferred embodiments provided by the invention are methods of using the stimulatory pan DR peptides, optionally with an adjuvant component in any vaccine formulation to enhance an immune response against an administered immunogen. For example, the pan DR binding peptides can be admixed with or conjugated with a CTL-inducing peptide and administered to induce a CTL response against, e.g., virally infected or tumor-associated antigen-bearing cells. In another embodiment, the pan DR peptides are conjugated with antibody-inducing peptides or carbohydrates. In addition, the pan DR peptides can be admixed with an antibody-inducing peptide or carbohydrate. The use of helper peptides to enhance antibody responses against particular determinants is described in, e.g., Hervas-Stubbs et al., Vaccine 12:867-871 (1994).

The one or more CTL and/or antibody-inducing peptides or other antigens can be administered with one or more pan DR peptides in a mixture that may or may not involve noncovalent associations between the peptides and/or other antigens. For instance, one or more of the peptides may be lipidated.

Alternatively, the peptides can be covalently linked to form a PADRE®-antigenic determinant. To facilitate the association of the antigenic determinant with the PADRE® peptide, additional amino acids can be added to the termini of the peptides. The additional residues can also be used for coupling to a carrier, support or larger peptide, for reasons discussed herein, or for modifying the physical or chemical properties of the peptide or oligopeptide, or the like. Amino acids such as tyrosine, cysteine, lysine, glutamic or aspartic acid, or the like, can be introduced at the C— or N-terminus of the peptide or oligopeptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxy amidation, e.g., ammonia, methylamine, etc. In some instances these modifications may provide sites for linking to a support or other molecule.

Accordingly, the CTL- or antibody-inducing peptide, carbohydrate, or other antigen can be linked to the pan DR binding peptide either directly or via a spacer either at the amino or carboxy terminus of the CTL peptide. The amino terminus of either the CTL- or antibody-inducing peptide or the pan DR binding peptide may be acylated to facilitate linkage. For example, the conjugate of a CTL or antibody-inducing antigen which is linked to the pan DR peptide can in turn be linked to certain alkanoyl (C₁-C₂₀) lipids via one or more linking residues such as Gly, Gly-Gly, Ser, Ser-Ser as described below.

In particularly preferred embodiments, CTL- or antibody-inducing peptides or carbohydrate epitope/pan DR binding conjugates of the invention are linked by a spacer molecule. See, e.g., copending U.S. patent application Ser. No. 08/788,822, filed Jan. 23, 1997. Alternatively, the peptide or other antigen can be linked to the pan DR binding peptide without a spacer. The spacer is typically comprised of relatively small, neutral molecules, such as amino acids or amino acid mimetics, which are substantially uncharged under physiological conditions and may have linear or branched side chains, and also natural amino (butyric, caproic, hexanoic, octanoic, lauric, or palmitic) acid. The spacers are typically selected from, e.g., Ala, Gly, or other neutral spacers of nonpolar amino acids or neutral polar amino acids. In certain preferred embodiments herein the neutral spacer is Ala. It will be understood that the optionally present spacer need not be comprised of the same residues and thus may be a hetero- or homo-oligomer. Preferred exemplary spacers are homo-oligomers of Ala and amino caproic acid. When present, the spacer will usually be at least one or two residues, more usually three to 16 residues. In other embodiments the pan DR binding peptide is conjugated to the CTL- or antibody-inducing peptide, carbohydrate, or other antigen, preferably with the pan DR binding peptide positioned at the amino terminus. The two moieties can be joined by a neutral linker, such as Ala-Ala-Ala or the like. Moreover, a pan DR binding peptide can be linked to another molecule by means of a spacer which is a surface active molecule such as a lipid or surfactant.

Where the same peptide is linked to itself, thereby forming a homopolymer, a plurality of repeating epitopic units is presented. For example, multiple antigen peptide (MAP) technology can be used to construct polymers containing both CTL and/or antibody peptides and PADRE® peptides. When the peptides differ, e.g., a cocktail representing different viral subtypes, different epitopes within a subtype, different HLA restriction specificities, or peptides which contain T helper epitopes, heteropolymers with repeating units are provided. In addition to covalent linkages, noncovalent linkages capable of forming intermolecular and intrastructural bonds are also contemplated.

In some embodiments it may be desirable to include in the pharmaceutical compositions of the invention at least one component which assists in priming CTL. For example, one or more surface-active molecules can be included in the compositions. These compounds include, for example, lipids, polymers (e.g., nonionic block copolymers), polyalkylene glycol (e.g., polyethylene glycol) and surfactants. The inclusion in the compositions of such compounds, which can cause the peptides to become membrane-bound, can alter how the peptide is delivered to cells or tissues and can also affect clearance and/or degradation.

Lipids are one example of components that have been identified as agents capable of assisting the priming of CTL in vivo against viral antigens. For example, palmitic acid residues can be attached to the alpha and epsilon amino groups of a Lys residue and then linked, e.g., via one or more linking residues such as Gly, Gly-Gly-, Ser, Ser-Ser, or the like, to an immunogenic peptide. The lipidated peptide can then be injected directly in a micellar form, incorporated into a liposome or emulsified in an adjuvant, e.g., incomplete Freund's adjuvant. In a preferred embodiment a particularly effective immunogen comprises palmitic acid attached to alpha and epsilon amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinly-seryl-serine (P₃CSS) can be used to prime virus specific CTL when covalently attached to an appropriate peptide. See, Deres et al., Nature 342, 561-564 (1989). Peptides of the invention can be coupled to P₃CSS, for example, and the lipopeptide administered to an individual to specifically prime a CTL response to the target antigen. Further, as the induction of neutralizing antibodies can also be primed with P₃CSS conjugated to a peptide which displays an appropriate epitope, the two compositions can be combined to more effectively elicit both humoral and cell-mediated responses to an infection or tumor.

The antibody-inducing or CTL-inducing antigen (e.g., a polysaccharide, protein, glycoprotein, lipid, glycolipid, lipopolysaccharide, and the like) that is administered in conjunction with the pan DR peptides of the invention can be an antigen that is associated with, e.g., a pathogen, a diseased cell, etc. For example, the antigen can be from a bacterium, a virus, a cancer cell, a fungus, or a parasite, among others. Peptide and carbohydrate epitopes from a large number of antigenic biomolecules can also be used in the conjugates and compositions of the present invention. For a listing of suitable antigens for use in the present invention, see, e.g., BioCarb Chemicals Catalogue; and The Jordan Report: Accelerated Development of Vaccine 1995 NIH, Bethesda, Md., 1995). Some examples of suitable antigens are described below.

CTL- and Antibody-Inducing Peptides.

CTL and/or antibody-inducing peptides can be administered with the pan DR peptides of the invention to enhance an immune response. CTL and antibody epitopes from a number of antigenic proteins can be used in the conjugates of the present invention. Examples of suitable antigens include prostate specific antigen (PSA), hepatitis B core and surface antigens (HBVc, HBVs), hepatitis C antigens, Epstein-Barr virus antigens, human immunodeficiency virus (HIV) antigens and human papilloma virus (HPV) antigens. An exemplary viral antigen includes those derived from HIV (e.g., gp120). Exemplary fingal antigens include those derived from Candida albicans, Cryptococcus neoformans, Coccidoides spp., Histoplasma spp., and Aspergillus spp. Parasitic antigens include those derived from Plasmodium spp., trypanosoma spp., Schistosoma spp., Leishmania spp., and the like. Additionally, examples of tumor-associated antigens include carcinoembryonic antigen, p53, melanoma antigens (e.g., MAGE-1, -2, or -3), HER2/neu, or prostate-related antigens (e.g., PSA).

In certain embodiments the CTL peptides of the invention are derived from within the HBV surface antigen or the nucleocapsid polypeptides, core and precore. In more preferred embodiments described herein the CTL-inducing peptides are derived from the region of HBenv₃₀₉₋₃₂₈ (peptide 799.08), HBenv₃₂₉₋₃₄₈ (peptide 799.09), HBenv₃₄₉₋₃₆₈ (peptide 799.10), or the region HBc₉₁₋₁₁₀ (peptide 802.03), where the numbering is according to Galibert et al., Nature 281, 646 (1979).

CTL- and antibody-inducing peptides, and how such peptides are obtained, are described in more detail in U.S. patent application Ser. No. 08/485,218, filed Jun. 7, 1995.

CTL- and Antibody-Inducing Carbohydrate Epitopes.

Carbohydrate epitopes, both antibody-inducing and CTL-inducing, from a wide variety of sources can be used in conjunction with the pan DR peptides of the invention. For example, carbohydrate epitopes associated with many types of cancer, or infectious agents (e.g., bacteria, fungi, parasites, viruses, and the like), are described in more detail in U.S. patent application Ser. No. 08/788,822, filed Jul. 23, 1997.

Examples of suitable antigens include those derived from bacterial surface polysaccharides and which can be used in carbohydrate-based vaccines. Bacteria typically express carbohydrate epitopes on their cell surface, including glycoproteins, glycoplipids, O-specific side chains of lipopolysaccharides, capsular polysaccharides and the like. Exemplary bacterial strains include Streptococcus pneumonia, Neisseria meninitidis, Haemophilus influenza, Klebsiella spp., Pseudomonas spp., Salmonella spp., Shigella spp., and Group B streptococci. A number of suitable bacterial carbohydrate epitopes are described in the art (see, e.g., Sanders et al. Pediatr. Res. 37:812-819 (1995); Bartoloni et al. Vaccine 13:463-470 (1995); Pirofski et al., Infect. Immun. 63:2906-2911 (1995) and International Publication No. WO 93/21948). Examples of suitable tumor carbohydrate antigens include GM2, GD2, GD3, Globo H, Le^{y}, Sialyl Le^{a}, T epitope, T_{N} epitope, ST_{N} epitope as described in the art (see, e.g., Livingston et al., Cancer Immunol. Immununother. 45:1-9 (1997)).

D. Pharmaceutical Compositions

The compounds of the present invention, and pharmaceutical and vaccine compositions thereof, can be administered to mammals, particularly humans, for prophylactic and/or therapeutic purposes. In preferred embodiments, the present invention is used to elicit and/or enhance immune responses against immunogens. For instance, CTL peptide-pan DR peptide mixtures may be used to treat and/or prevent viral infection or cancer. Alternatively, carbohydrate immunogens can be used. Examples of diseases which can be treated using the present invention include various bacterial infections, viral infections, fingal infections, parasitic infections and cancer. In other embodiments, inhibitory pan DR peptides of the invention are administered to block an inappropriate or otherwise undesirable immune response (e.g., autoimmunity and the like).

In therapeutic applications, the present invention is administered to an individual already suffering from an inappropriate immune response, cancer, or infected with the pathogen of interest. Those in the incubation phase or the acute phase of the disease can be treated with the present invention separately or in conjunction with other treatments, as appropriate.

In therapeutic applications, a composition of the present invention is administered to a patient in an amount sufficient to block an undesired immune response, or to elicit an effective CTL response or humoral response to the microorganism or tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician.

Therapeutically effective amounts of the compositions of the present invention generally range for the initial immunization that is for therapeutic or prophylactic administration, from about 1.0 μg to about 10,000 μg of peptide for a 70 kg patient, usually from about 100 to about 8000 μg, and preferably between about 200 and about 6000 μg. These doses are followed by boosting dosages of from about 1.0 μg to about 1000 μg of peptide pursuant to a boosting regimen over weeks to months depending upon the patient's response and condition as determined by measuring specific immune responses.

It must be kept in mind that the compositions of the present invention may generally be employed in serious disease states, that is, life-threatening or potentially life threatening situations. In such cases, in view of the minimization of extraneous substances and the relative nontoxic nature of the conjugates, it is possible and may be felt desirable by the treating physician to administer substantial excesses of these compositions.

Further, the present invention can be used prophylactically to prevent and/or ameliorate inappropriate immune responses, bacterial infections, viral infections, fungal infections, parasitic infections or cancer. Effective amounts are as described above. Additionally, one of ordinary skill in the vaccine arts understands how to adjust or modify prophylactic treatments, as appropriate, for example by boosting and adjusting dosages and dosing regimes in accordance with known immunology and pharmacology principles.

Therapeutic administration may begin at the first sign of disease or the detection or surgical removal of tumors or shortly after diagnosis in the case of acute infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, the composition can be targeted to them, minimizing need for administration to a larger population.

The present invention is also be used for the treatment of chronic infection and to stimulate the immune system to eliminate, e.g., virus-infected cells in carriers. It is important to provide compositions of the present invention in an amount, in a formulation and via a mode of administration sufficient to effectively elicit and/or enhance an immune response. Thus, for treatment of chronic infection, a representative dose is in the range of about 1.0 μg to about 5000 μg, preferably about 5 μg to 1000 μg for a 70 kg patient per dose. Immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the viral infection has been eliminated or substantially abated and for a period thereafter.

The pharmaceutical compositions for therapeutic or prophylactic treatment are intended for parenteral, topical, oral or local administration. Typically, the pharmaceutical compositions are administered parenterally, e.g., intravenously, subcutaneously, intradermally, or intramuscularly. The vaccine compositions of the invention are particularly suitable for oral administration, due to their ease of administration. Thus, the invention provides compositions for parenteral administration which comprise a solution of the peptides or conjugates dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate and the like.

Carriers can be used in combination with peptides of the invention. Carriers are well known in the art, and include, e.g., thyroglobulin, albumins such as bovine serum albumin, tetanus toxoid, polyamino acids such as poly(lysine:glutamic acid), hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, or alum are materials well known in the art.

The concentration of compositions of the present invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The present invention may also be administered via liposomes, which serve to target the conjugates to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the composition to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired composition of the present invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Bioshys. Bioeng. 9, 467 (1980), U.S. Pat. Nos. 4,235,871, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension containing a composition of the present invention may be administered intravenously, locally, topically, etc. in a dose which varies according to, inter alia, the manner of administration, the composition being delivered, and the stage of the disease being treated.

Alternatively, DNA or RNA encoding one or more PADRE® peptides and a polypeptide containing one or more CTL epitopes or antibody-inducing epitopes may be introduced into patients to obtain an immune response to the polypeptides which the nucleic acid encodes. Wolff et. al., Science 247: 1465-1468 (1990) describes the use of nucleic acids to produce expression of the genes which the nucleic acids encode.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the compositions of the present invention are preferably supplied in finely divided form along with a surfactant and propellant. Typical percentages of the composition are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

As noted herein, compositions of the invention may be introduced into a host, including humans, linked to its own carrier or as a homopolymer or heteropolymer of active peptide units. Such a polymer has the advantage of increased immunological reaction and, where different peptides are used to make up a hetero polymer, the ability to induce antibodies and/or CTLs that react with different antigenic determinants.

And, as mentioned above, immune responses can be primed by conjugating compositions of the present invention to lipids, such as P₃CSS. Upon immunization with a composition as described herein, via injection, aerosol, oral, transdermal or other route, the immune system of the host responds by producing an enhanced immune response, humoral and/or cellular.

In some instances it may be desirable to combine the compositions of the present invention with other modalities, such as vaccines which induce neutralizing antibody responses to infections and cancers of interest.

The peptides of this invention can also be used to make monoclonal antibodies. Such antibodies are useful as diagnostic or therapeutic agents.

The peptides of the invention are also used as diagnostic reagents. For example, a peptide of the invention can be used to determine the susceptibility of a particular individual to a treatment regimen which employs the peptide or related peptides, and thus can be helpful in modifying an existing treatment protocol or in determining a prognosis for an affected individual. In addition, the peptides can also be used to predict which individuals will be at substantial risk for developing chronic infection.

E. Construction of Expression Vectors for Nucleic Acids Encoding Pan DR Binding Peptides and Administration In Vivo

The peptides of the invention can be delivered by way of nucleic acids (“minigenes”) through the construction of expression vectors encoding a peptide epitope of interest. Such vectors will contain at least one promoter element that is capable of expressing a transcription unit encoding at least one pan DR binding peptide and an MHC targeting sequence for the appropriate cells of an organism, so that the antigen is expressed and targeted to the appropriate MHC molecule. For example, if the expression vector is administered to a mammal such as a human, a promoter element that functions in a human cell is incorporated into the expression vector. The vectors may also optimally include nucleic acid sequences encoding multiple pan DR binding peptides and/or one or more MHC class I epitopes.

Routine techniques in the field of recombinant genetics may be used to construct the expression vectors. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994); Oligonucleotide Synthesis: A Practical Approach (Gait, ed., 1984); Kuijpers, Nucleic Acids Research 18(17):5197 (1994); Dueholm, J. Org. Chem. 59:5767-5773 (1994); Methods in Molecular Biology, volume 20 (Agrawal, ed.); and Tijssen, Laboratory Techniques in Biochemistry and Molecular Biology—Hybridization with Nucleic Acid Probes, e.g., Part I, chapter 2 “Overview of principles of hybridization and the strategy of nucleic acid probe assays” (1993)).

The minigenes can comprise one or many different epitopes. The nucleic acid encoding the epitopes are assembled in a minigene according to standard techniques. In general, the nucleic acid sequences encoding minigene epitopes are isolated using amplification techniques with oligonucleotide primers, or are chemically synthesized. Recombinant cloning techniques can also be used when appropriate. Oligonucleotide sequences are selected which either amplify (when using PCR to assemble the minigene) or encode (when using synthetic oligonucleotides to assemble the minigene) the desired epitopes.

Amplification techniques using primers are typically used to amplify and isolate sequences encoding the epitopes of choice from DNA or RNA (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify epitope nucleic acid sequences directly from mRNA, from cDNA, from genomic libraries or cDNA libraries. Restriction endonuclease sites can be incorporated into the primers. Minigenes amplified by the PCR reaction can be purified from agarose gels and cloned into an appropriate vector.

Synthetic oligonucleotides can also be used to construct minigenes. This method is performed using a series of overlapping oligonucleotides, representing both the sense and non-sense strands of the gene. These DNA fragments are then annealed, ligated and cloned. Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The epitopes of the minigene are typically subcloned into an expression vector that contains a strong promoter to direct transcription, as well as other regulatory sequences such as enhancers and polyadenylation sites. Suitable promoters are well known in the art and described, e.g., in Sambrook et al. and Ausubel et al. Eukaryotic expression systems for mammalian cells are well known in the art and are commercially available. Such promoter elements include, for example, cytomegalovirus (CMV), Rous sarcoma virus LTR and SV40.

The expression vector typically contains a transcription unit or expression cassette that contains all the additional elements required for the expression of the minigene in host cells. A typical expression cassette thus contains a promoter operably linked to the minigene and signals required for efficient polyadenylation of the transcript. Additional elements of the cassette may include enhancers and introns with functional splice donor and acceptor sites.

In addition to a promoter sequence, the expression cassette can also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic cells may be used. Expression vectors containing regulatory elements from eukaryotic viruses are typically used in eukaryotic expression vectors, e.g., SV40 vectors, papilloma virus vectors, and vectors derived from Epstein Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells. In one embodiment, the vector pEP2 is used in the present invention.

Other elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

The invention further relates to methods of administering a pharmaceutical composition comprising an expression vector of the invention to stimulate an immune response. The expression vectors are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997).

A pharmaceutical composition comprising an expression vector of the invention can be administered to stimulate an immune response in a subject by various routes including, for example, orally, intravaginally, rectally, or parenterally, such as intravenously, intramuscularly, subcutaneously, intraorbitally, intracapsularly, intrathecally, intraperitoneally, intracistemally or by passive or facilitated absorption through the skin using, for example, a skin patch or transdernal iontophoresis, respectively. Furthermore, the composition can be administered by injection, intubation or topically, the latter of which can be passive, for example, by direct application of an ointment or powder, or active, for example, using a nasal spray or inhalant. An expression vector also can be administered as a topical spray, in which case one component of the composition is an appropriate propellant. The pharmaceutical composition also can be incorporated, if desired, into liposomes, microspheres or other polymer matrices (Felgner et al., U.S. Pat. No. 5,703,055; Gregoriadis, Liposome Technology, Vols. I to III (2nd ed. 1993), each of which is incorporated herein by reference). Liposomes, for example, which consist of phospholipids or other lipids, are nontoxic, physiologically acceptable and metabolizable carriers that are relatively simple to make and administer.

The expression vectors of the invention can be delivered to the interstitial spaces of tissues of an animal body (Felgner et al., U.S. Pat. Nos. 5,580,859 and 5,703,055). Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration typically involves mechanically or chemically irritating the outermost layer of epidermis to stimulate an immune response to the irritant (Carson et al., U.S. Pat. No. 5,679,647).

Other effective methods of administering an expression vector of the invention to stimulate an immune response include mucosal administration (Carson et al., U.S. Pat. No. 5,679,647). For mucosal administration, a presently preferred method of administration comprises intranasal administration of an appropriate aerosol containing the expression vector and a pharmaceutical composition. Suppositories and topical preparations are also effective for delivery of expression vectors to mucosal tissues of genital, vaginal and ocular sites. Additionally, expression vectors can be complexed to particles and administered by a vaccine gun.

The dosage of a minigene construct to be administered is dependent on the method of administration and will generally be between about 0.1 μg up to about 200 μg. For example, the dosage can be from about 0.05 μg/kg to about 50 mg/kg, in particular about 0.005-5 mg/kg. An effective dose can be determined, for example, by measuring the immune response after administration of an expression vector. For example, the production of antibodies specific for the MHC class II epitopes or MHC class I epitopes encoded by the expression vector can be measured by methods well known in the art, including ELISA or other immunological assays. In addition, the activation of T helper cells or a CTL response can be measured by methods well known in the art including, for example, the uptake of ³H-thymidine to measure T cell activation and the release of ⁵¹Cr to measure CTL activity.

### EXAMPLES

The following examples are offered to illustrate, but not to limit the present invention.

### Example 1

### Pan DR Binding Sequence Provides T-Cell Help for Induction of Protective Antibodies Against Plasmodium yoelli sporozoites

1. Introduction

Pan DR epitopes (PADRE®) as disclosed herein bind to most common HLA DR molecules. The pan DR peptides are immunogenic for human T cells, as described herein and in Alexander et al., Immunity 1:751-61 (1994). PADRE® peptides fortuitously also bind the mouse MHC molecules, IA^{b}, thus providing a test system for demonstrating that that they deliver powerful helper T-cell activity in vivo, as demonstrated by enhancement of specific responses directed against an influenza virus-derived class I-restricted epitope.

The capacity of PADRE® epitopes to deliver help for antibody responses in vivo is also described herein, and has been subsequently published (Del Guercio et al., Vaccine 15:441-8 (1997)). According to a commonly held view, efficient induction of antibody responses requires large multivalent antigens, which can fulfill the requirements for cross-linking of surface Ig on the surface of specific B-cells. Consequently it was assumed that single, monovalent antigens are not efficient immunogens for B-cell responses. However, the peptides of the present invention, which include short, linear, monovalent constructs that contain the PADRE® epitope, are remarkably active immunogens in vivo, in terms of induction of specific antibody responses.

In one demonstration of this antibody-inducing activity, the Plasmodium vivax circumsporozoite protein B-cell epitope (PvB), previously shown to be exquisitely T-cell dependent (Nardin et al., Eur J. Immunol 17:1763-7 (1987)), was incorporated in 33-residue long peptide immunogen. This construct induced responses almost exclusively composed of IgG, long lasting and indistinguishable in titer from those induced by more complex multivalent branched chain polymer multiple antigenic peptide (MAP) and KLH conjugates (Del Guercio et al., Vaccine 15:441-8 (1997)). The efficacy of this type of single monovalent construct was generalized to B-cell epitopes derived from other parasites (P. yoelii and P. falciparum). Resulting antibodies were shown to bind intact sporozoites, thus suggesting that these responses were of potential biological relevance (Del Guercio, M. F. et al., Vaccine 15:441-8 (1997)).

This Example demonstrates that the antibodies induced by the P. falciparum and P. yoelii CSP-PADRE® peptides inhibit sporozoite invasion of hepatocytes in vitro, and that immunization with the PyCSP-PADRE® linear peptide protects mice against challenge with P. yoelii sporozoites comparably to the PyCSP-MAP (Franke et al., Vaccine 17:1201-1205 (1999)).

2. Methods

2.1. Mice

Four to six week old female C57BL/6 mice were purchased from Jackson Laboratories (Bar Harbor, Me.).

2.2. Immunogens

2.2.1 Linearpeptides

Peptides encompassing B-cell epitopes from the central immunodominant circumsporozoite repeat region of circumsporozoite proteins (CSP) of P. yoelii (PyB) or P. falciparum (PfB) were synthesized by standard MOC chemistry, purified by HPLC and their purity and identity verified by HPLC and mass spectrometry. Sequences: PyB=G(QGPGAP)₄ (Charoenvit, Y. et al., J. Immunol. 146:1020-5 (1991)); PfB=(NANP)₄ (Nussenzweig, V. et al., Adv Immunol 45:283-334 (1989); Dame, J. B. et al., Science 225:593-9 (1984)). Peptides colinearly synthesized to encompass PADRE® were also produced using the same methods. PADRE®-PfB sequence: aKXVAAWTLKAa(NANP)₄GGS; PADRE®-PyB sequence: aKXVAAWTLKAa(QGPGAP)₄GGS.

2.2.2. PyCSP-MAP

A multiple antigen peptide (PyCSP-MAP) was also synthesized as previously described (Wang, R. et al., J. Immunol 154:2784-93 (1995); Valmori, D. et al., J. Immunol Meth 149:717-21 (1992)). In brief, it included a lysine core and four branches. Each branch included four copies of the protective B-cell epitope, QGPGAP, from the PyCSP and the universal T-helper epitopes from tetanus toxin, p2p30 (p2=QYIKANSKFIGITE; p30=FNNFTVSFWLRVPKVSASHLE) (Wang, R. et al., J. Immunol 154:2784-93 (1995)).

2.3 Immunization

In certain experiments, peptides were emulsified in complete Freund's adjuvant (CFA) and injected subcutaneously (S.C.) at the base of the tail in 100 μl volume containing 100 μg of peptide. Mice were boosted with 100 μg of peptide emulsified in 100 μl of incomplete Freund's adjuvant (IFA) 1 month later. Mice were bled just prior to boosting, and 2 weeks after boosting.

In other experiments, the peptides were suspended in phosphate buffered saline (PBS) and emulsified in TiterMax™ (CytRx Corporation, Norcross, Ga.). Mice were immunized with peptides by S.C. injection at the base of the tail three times at 3-week intervals. The injection volume was 25 μl. The doses and regimens for these immunizations were based on those determined to be optimal from previous experiments (Del Guercio, M. F. et al., Vaccine 15:441-8 (1997); Wang, R. et al., J. Immunol 154:2784-93 (1995)) (Sette et al., unpublished observations).

2.4 Cell Culture

Mouse hepatocytes were obtained by in situ collagenase perfusion as previously described (Charoenvit, Y. et al., Exp Parasitol 80:419-29 (1995)). Cells were plated at a concentration of 1×10⁵ cells/well in 8-well chamberslides (Nunc, Naperville, Ill.) using Complete Medium (MEM with Earle's balanced salts, supplemented with 0.2% BSA, 10% fetal calf serum, 2% penicillin-streptomycin, 2 mg insulin, 1% glutamine and 1% nonessential amino acids solution). All cell culture reagents were obtained from quality Biological, Gaithersburg, Md. The slides were incubated overnight at 37° C. in a 5% CO₂/95% air environment. The medium was changed the following day and fresh medium containing dexamethasone (7×10⁻⁵) was added to the cultures.

Human hepatocytes were isolated from nontransplantable liver tissue donated for research. A single cell suspension was made by a two-step collagenase perfusion as previously reported (Mellouk, S. et al., Bull World Health Organ 68 (Suppl):52-9 (1990)). The cells were seeded onto 8-well chamberslides (Nunc, Naperville, Ill.) at a concentration of 1.25×10⁵ cells/well in Complete Medium. The hepatocytes were allowed to attach and spread overnight with the media being changed the following day.

2.4.1. Parasites

The 17XNL (nonlethal) strain of P. yoelii or the NF54 strain of P. falciparum were used for murine and human hepatocyte in vitro infections, respectively. Parasites were harvested using a modification of the Ozaki technique for the rapid isolation of sporozites (Ozaki, L. S. et al., J. Parasitol 70:831-3 (1984)).

2.5. Inhibition of Liver Stage Development Assay (ILSDA)

The capacity of sera from immunized mice to inhibit sporozoite invasion and development in hepatocytes was assessed by ILSDA as previously described (Wang, R. et al., J. Immunol 154:2784-93 (1995); Charoenvit, Y. et al., J. Immunol. 146:1020-5 (1991)). Briefly, 25 μl of the serum samples, diluted to 2× their final concentration, were added to triplicate chamber-slide wells. P. yoeilli or P. falciparum sporozoites, 75,000/well, were then added in a volume of 25 μl to the mouse or human hepatocyte cultures respectively. The cultures were incubated for 3 h, then washed extensively with fresh medium to remove unattached sporozoites. The infected hepatocytes were then incubated for a further 2 (P. yoelii) or 5 days (P. falciparum) with the medium being changed each day of culture. The slides were fixed and immunostained with either a P. yoelii- or P. falciparum-specific monoclonal antibody. Schizonts were counted from the triplicate cultures and the percent inhibition determined as follows: control−test/control×100. Sera from mice injected with CFA (first dose) and IFA (second and third doses) served as the negative controls.

2.6. Antibodies to Sporozoites, Synthetic Peptide and Recombinant CSP Protein

Mice were bled two weeks after the last immunization. Antibody titers to air-dried sporozoites were determined by the indirect fluorescent antibody test (IFAT) against air-dried P. yoelii sporozoites as described previously (Wang, R. et al., J. Immunol 154:2784-93 (1995); Charoenvit, Y. et al., J. Immunol. 146:1020-5 (1991)). Results were reported in units, defined as reciprocals of the serum dilution at which the OD reading (wavelength 410 nm) is 1.0.

2.7. Challenge with Sporozoites

P. yoelii 17XNL (nonlethal strain), clone 1.1 sporozoites, which were hand dissected from the salivary glands of Anopheles stephensi, and suspended in Medium 199 containing 5% fetal bovine serum were used for the challenge. Mice were challenged by intravenous injection of 50 sporozoites in a volume of 0.2 ml. Mice were monitored for blood stage infection from day 5 to day 14 after injection of sporozoites by examining Giemsa-stained thick blood smears. In our experience, mice that are negative on day 14 never become positive, so mice negative on day 14 were considered protected.

3. Results and Discussion

3.1. Inhibition of Sporozoite Invasion of Hepatocytes

The antibody levels after administration to C57BL/6 (H-2^{b}) mice of 2 doses of PADRE®-PfB and PADRE®-PyB peptides in CFA/IFA have now been described (see, e.g., Del Guercio, M. F. et al., Vaccine 15:441-8 (1997)). As a foundation for moving to in vivo challenge studies we determined whether the antibodies induced by this immunization regimen inhibited sporozoite invasion of hepatocytes in vitro. We first assessed the effects of immune sera on invasion and development of P. falciparum sporozoites in human hepatocytes. Sera from mice immunized with PyB or PADRE®-PyB did not inhibit sporozoite invasion and development (Table 6). The greatest inhibition was by sera from mice immunized with PADRE®-PfB (97%). Sera from mice immunized with PfB also gave significant in vitro inhibition (80%). This was probably due to the fact that PfB also contains a helper T-cell epitope for H-2^{b} mice (Good, M. F. et al., J Exp Med 164:655-60 (1986); Hoffman, S. L. et al., Exp Parasitol 64:64-70 (1987)) and did not require PADRE® for induction of antibodies, although the PADRE®-PfB-immunized mice did produce higher levels of antibodies, as previously shown (Del Guercio, M. F. et al., Vaccine 15:441-8 (1997)).

TABLE 6
In vitro inhibition of liver stage development in human
hepatocytes infected with P. falciparum sporozoites.

| Sera^{a} | Mean schizonts/well ± S.D. | % Inhibition |
|---|---|---|
| CFA | 77 ± 4 | n.c.^{b} |
| PyB | 70 ± 14 | 9.1 |
| PADRE ®-PyB | 84 ± 8.5 | −9.1 |
| PfB | 15.6 ± 8.7 | 79.7 |
| PADRE ®-PfB | 2 ± 0.8 | 97.4 |

^{a}Sera were assessed at a final dilution of 1:100.
^{b}n.c. designates the negative control.

In the case of P. yoelii sporozoites, sera from mice immunized with PfB or PADRE®-PfB gave minimal, nonsignificant inhibition (8.5-13.4% at a serum dilution of 1:100) of invasion of mouse liver cells (Table 7). Sera from mice immunized with PyB gave 50% inhibition at this dilution, while mice immunized with PADRE® PyB gave significantly greater inhibition (91 and 88% at 1:100 and 1:200 dilutions) (Table 7).

In conclusion, the data in Table 6 and Table 7 clearly demonstrate that sera from mice immunized with PADRE®-PFB inhibited P. falciparum sporozoite invasion of human hepatocytes by 98% and had no significant effect on P. yoelii invasion of murine hepatocytes. In contrast, sera from mice immunized with PADRE®-PyB inhibited P. yoelii sporozoite invasion and development in murine hepatocytes by approximately 90%, but had no significant effect on P. falciparum invasion and development in human hepatocytes.

TABLE 7
In vitro inhibition of liver stage development in mouse
hepatocytes infected with P. yoelii sporozoite

| Sera^{a} | Mean schizonts/well + S.D. | % Inhibition |
|---|---|---|
| CFA | | |
| 1:100 | 82 + 3.3 | ^{a}n.c. |
| 1:200 | 81 + 10 | n.c. |
| PyB | | |
| 1:100 | 41 + 3.8 | 50.0 |
| 1:200 | 46 + 4 | 43.2 |
| PADRE ®-PyB | | |
| 1:100 | 7.6 + 1.2 | 90.7 |
| 1:200 | 96 + 2 | 88.1 |
| PfB | | |
| 1:100 | 71 ± 7 | 13.4 |
| 1:200 | 76 ± 7 | 6.2 |
| PADRE ®-PfB | | |
| 1:100 | 75 ± 5 | 8.5 |
| 1:200 | 79 ± 6 | 2.5 |

^{a}n.c. designates the negative control.

3.2. PADRE®-PyB Constructs Protect Against Sporozoite Challenge

Encouraged by the data from the experiments shown above, we determined next if immunization with the PADRE®-PyB peptide would protect mice against sporozoite challenge. In order to select a control immunogen we relied on the following information. We have previously reported that immunization with a multiple antigen peptide branched chain polymer including the 35 amino acid P2P30 universal T-cell epitope sequences from tetanus toxin, and four copies of the six amino acid tandem repeat (QGPGAP) from the P. yoelii circumsporozoite protein (PyCSP) in multiple adjuvants induces high levels of antibodies that inhibit sporozoite invasion of hepatocytes in vitro and protect against sporozoite challenge in vivo (Wang, R. et al., J. Immunol 154:2784-93 (1995)). We have also determined that doses of 25 μg of this PyCSP MAP induce higher levels of protection than do higher doses. Accordingly, this immunogen was used as a positive control in our experiments.

The data shown in Table 8 demonstrate that immunization with 100 μg of the linear PADRE®-PyB peptide induced levels of antibodies against sporozoites and the PyCSP at least as high as those induced in mice that received 25 μg of the PyCSP-MAP. Immunization with PyB alone was poorly immunogenic. Most importantly, immunization with the simple linear PADRE®-PyB peptide in Titermax™ protected 75% of mice against developing blood stage parasitemia after sporozoite challenge. This level of protection was comparable to that induced by immunization with the more complex, and difficult to synthesize and characterize, PyCSP-MAP administered in Titermax™.

Herein, we have demonstrated in the murine model of malaria, represented by infection with P. yoelii sporozoites, and the efficacy of simple linear constructs containing dominant B-cell epitopes and PADRE® peptides. This type of construct has previously been shown to be highly immunogenic in mice, utilizing various different B-cell epitopes (Del Guercio, M. F. et al., Vaccine 15:441-8 (1997)). The antibody response elicited was indistinguishable in magnitude, isotype composition and duration from responses obtained utilizing the same B-cell epitopes in the context of much more complex immunogens such as KLH conjugates, or MAP constructs. Alum or ISA51 adjuvants were effective in delivering these simple PADRE® constructs, underlining the potential for further preclinical and clinical development. The potential for clinical development is further underlined by the fact that more complex immunogens, although highly immunogenic and efficacious in model systems, appear to face significant challenges to further development, due to difficulties in analytical and manufacturing processes.

Of note, a P. falciparum CSP-MAP including P. falciparum B— and T-cell epitopes has been utilized in clinical trials (de Oliveira, G. A. et al., Vaccine 12:1012-7 (1994); Moreno, A. et al., J Immunol 151:489-99 (1993); Calvo Calle, J. M. et al., J Immunol 150:1403-12 (1993)). Recently a number of methods have been proposed for simplifying the synthesis and characterization of MAPs (Rose, K. et al., Mol Immunol 32:1031-7 (1995); Zeng, W. et al., J Pept Sci 2:66-72 (1996)). However, the data reported herein and our preliminary data in monkeys, indicate that MAPs and linear synthetic peptides with P. falciparum B-epitopes and PADRE® or P2P30 as T-helper epitopes induce nearly comparable antibody levels.

TABLE 8
Antibodies and protective immunity after immunization
of mice with PyCSP synthetic peptide vaccines

| | | | | ELISA | |
|---|---|---|---|---|---|
| (QGPGAP)₂ | | | | | |
| | | | IFAT | PyCS.1 | |
| Immunogen/ | Infected/ | % | Sporozoites | (OD Units × | |
| Adjuvant | challenged | Protected | (titer^{a} × 10 − 3) | 10 − 3)^{b} | |
| PyB/Titermax ™ | 7/8 | 12.5 | — | — | — |
| PADRE ®-PyB/ | 2/8 | 75.0 | 3.2 | 25.6 | 25.6 |
| Titermax ™ | | | | | |
| PyCSP-MAP/ | 2/7 | 71.4 | 3.2 | 12.8 | 12.8 |
| Titermax ™ | | | | | |
| —/Titermax ™ | 7/8 | 12.5 | — | — | — |
| Infectivity control | 8/8 | 0 | ND | ND | ND |

^{a}Titer is defined as the reciprocal of the last serum dilution yielding positive reactivity as detected by fluorescence microscopy.
^{b}The reciprocal of the serum dilution at which the optical density (410 nm) was 1.0.

### Example 2

### In Vitro Immunogenicity of Pan DR-Binding Peptides from Table 9

Introduction

To evaluate the potential of PADRE® molecules to provide T cell help, some of the preferred peptides set out in Table 9 were evaluated for their capacity to stimulate in vitro T cell responses in PBMC from 5 normal individuals.

Method

Generation of Antigen-Specific T cell Responses from Human PBMCs

PBMC from health donors were stimulated in vitro using a protocol adapted from Manca, F., Habeshaw, J. and Dalgleish, A., J. Immunol. 146, 1964-1971) (1991). PBMC were purified over Ficoll-Paque (Pharmacia LKB, Uppsala, Sweden) and plated in a 24-well tissue culture plate (Costar, Cambridge, Mass.) at 4×10⁶ PBMC/well. The peptides were added at a final concentration of 10 μg/ml. Cultures were then incubated at 37° C., 5% CO₂. On day 4, recombinant interleukin-2 (IL-2) was added at a final concentration of 10 ng/ml. Cultures were fed every 3 days thereafter by aspirating off 1 ml of media and replacing it with fresh medium containing IL-2. Two additional stimulations of the T cells with antigen were performed on approximately days 14 and 28. The T cells (3×10⁵/well) were stimulated with peptide (10 μg/ml) using autologous PBMC cells [2×10⁶ irradiated [7500 rads]/well] as antigen-presenting cells in a total of three wells of a 24-well tissue culture plate. In addition, on days 14 and 28, T cell proliferative responses were determined as follows: 2×10⁴ T cells/well; 1×10⁵ irradiated PBMC/well as antigen-presenting cells; peptide concentration varying between 0.01-10 μg/ml final concentration. The proliferation of the T cells was measured 3 days later by the addition of [³H]thymidine (1 μCi/well) (ICN, Irvine, Calif.) 18 hr prior to harvesting the cells. Cells were harvested onto glass filters (LKB Walla cell harvester 1295-001, Gaithersburg, Maryland), and thymidine incorporation (LKB β plate counter 1205) was measured.

TABLE 9
Binding Activity of PADRE ® Analogs

| | | | | | | | | | | | |
|---|---|---|---|---|---|---|---|---|---|---|---|
| | SEQ ID | | | | | | | | | | |
| PEPTIDE | NO. | SEQUENCE | DR1 | DR2wB2 | DR3 | DR4w4 | DR4w14 | DR5 | DR7 | DRw53 | DQ3.1 |

| | | | | | | | | | | | |
|---|---|---|---|---|---|---|---|---|---|---|---|
| 965.08 | 29 | aK(X)VAANTLKAAa-NH₂ | 1.2 | 3.8 | 250 | 3 | 13.8 | 8 | 192.3 | 163.8 | — |
| | | | (1) | | | | | | | | |
| 965.09 | 30 | aK(X)VAAYTLKAAa-NH₂ | 0.8 | 7.4 | 250 | 1 | 7 | 5.4 | 192.3 | 86.4 | — |
| 965.10 | 31 | aK(X)VAAWTLKAAa-NH₂ | 1.2 | 5.6 | 119 | 2.8 | 9.8 | 11.1 | 147.1 | 141.8 | 25 |
| 965.14 | 32 | aK(X)VAAKTLKAAa-NH₂ | 3.6 | 8 | 781 | 7.4 | 62.5 | 3.4 | 227 | 52.8 | — |
| 965.15 | 33 | aK(X)VAAHTLKAAa-NH₂ | 1.9 | 5.4 | 1389 | 3.2 | 13.8 | 29.9 | 156.3 | 79.2 | — |
| 965.16 | 34 | aK(X)VAAATLKAAa-NH₂ | 4.2 | 6.1 | 1471 | 6.2 | 55.6 | 16.7 | 227 | 131.9 | — |
| 965.17 | 35 | AK(X)VAAWTLKAAA-NH₂ | 2 | 5.9 | 1786 | 3.8 | 26.7 | 9.1 | 147.1 | 169.6 | — |
| 553.01 | 36 | QYIKANSKFIGITE | 51.5 | 20 | 2717 | 8036 | 10000 | 20 | 25 | — | — |
| 553.02 | 37 | qYIKANSKFIGITEa | 238 | 25.3 | — | — | — | 83.3 | 49 | — | — |
| | | | | | (2) | | | | | | |

(1) = nM IC₅₀ values
(2) dashes indicate >10,000 nM
(X) = cyclohexylalanine
“-NH₂” indicates amidation at the carboxyl terminus of the peptides.

Results and Discussion

The peptides 965.08, 965.09, 965.10, 965.14, 965.15, 965.16, and 965.17 were evaluated for their capacity to stimulate in vitro T cell responses in PBMCs from 5 normal individuals (X144, X162, X349, X372, and X460). See Table 10. After two in vitro stimulations only peptides 965.10 and 965.17 were able to stimulate T cells significantly, 3.9 and 2.4 times above background. However, by the third stimulation, all 7 peptides stimulated T cells at least 2 times above background. These results indicate that these pan DR epitopes provide T cell help for both humoral and cell-mediated responses.

TABLE 10
MAX CPM and STIMULATION INDEX

| | | | | | | | | | | | |
|---|---|---|---|---|---|---|---|---|---|---|---|
| X144 | | X162 | | X349 | | X372 | | X460 | | AVERAGE | |
| (Δmax | | (Δmax | | (Δmax | | (Δmax | | (Δmax | | ΔMAX | AVERAGE |
| cpm) | S.I. | cpm) | S.I. | cpm) | S.I. | cpm) | S.I. | cpm) | S.I. | CPM | S.I. |

2nd Stimulation

| | | | | | | | | | | | | |
|---|---|---|---|---|---|---|---|---|---|---|---|---|
| 965.08 | 8440 | 1.3 | 9234 | 2.6 | 9065 | 1.8 | 23017 | 1.6 | 6286 | 1.4 | 11208 | 1.7 |
| 965.09 | 13693 | 1.8 | 10710 | 1.1 | 18079 | 3.8 | 17813 | 1.6 | 0 | 1 | 12059 | 1.9 |
| 965.10 | 11663 | 1.8 | 20693 | 7.9 | 22133 | 3.5 | 44414 | 3.2 | 29933 | 3.3 | 25767 | 3.9 |
| 965.14 | 3570 | 1.1 | 4655 | 2.1 | 4421 | 1.3 | 10608 | 1.4 | 4365 | 1.4 | 5523 | 1.5 |
| 965.15 | 7179 | 1.4 | 8461 | 3.4 | 10848 | 1.8 | 10011 | 1.3 | 3100 | 1.2 | 7920 | 1.8 |
| 965.16 | 4388 | 1.2 | 4090 | 2.1 | 0 | 1 | 736 | 1 | 0 | 1 | 1843 | 1.3 |
| 965.17 | 17391 | 1.7 | 6323 | 2 | 26512 | 5.6 | 8471 | 1.3 | 4994 | 1.3 | 12738 | 2.4 |

3rd Stimulation

| | | | | | | | | | | | | |
|---|---|---|---|---|---|---|---|---|---|---|---|---|
| 965.08 | 3559 | 1.4 | 14622 | 2.3 | 31639 | 2.8 | 8710 | 1.9 | 1820 | 1.6 | 12070 | 2 |
| 965.09 | 14087 | 2.6 | 35635 | 5.2 | 31811 | 5.5 | 29159 | 4.4 | 2301 | 2.9 | 22599 | 4.1 |
| 965.10 | 33159 | 14 | 34548 | 11 | 38931 | 3.5 | 36995 | 15 | 11376 | 15 | 31002 | 11.5 |
| 965.14 | 4319 | 2.3 | 10734 | 1.7 | 17849 | 2 | 19140 | 2.1 | 874 | 2 | 10583 | 2 |
| 965.15 | 13055 | 2.4 | 12309 | 1.6 | 20653 | 2.4 | 30810 | 4.7 | 486 | 1.2 | 15462 | 2.5 |
| 965.16 | 5220 | 1.5 | 4762 | 1.9 | 9316 | 1.6 | 10439 | 1.7 | 37 | 1 | 5955 | 1.5 |
| 965.17 | 32665 | 4.1 | 28065 | 4.1 | 31983 | 4 | 30661 | 3.2 | 3619 | 2.2 | 25399 | 3.5 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A composition for enhancing an immune response to an immunogen, said composition comprising a nucleic acid segment encoding a pan DR binding oligopeptide of less than about 50 amino acid residues,
wherein said pan DR binding oligopeptide comprises a peptide of the formula R₁—R₂—R₃—R₄—R₅, wherein all amino acids of said peptide are L-amino acids, and wherein:
R₁ is any amino acid followed by alanine or lysine;
R₂ is selected from the group consisting of tyrosine and phenylalanine;
R₃ is 3 or 4 amino acids, wherein each amino acid is independently selected from the group consisting of alanine, isoleucine, serine and valine;
R₄ is selected from the group consisting of threonine-leucine-lysine, lysine-threonine, and tryptophan-threonine-leucine-lysine; and
R₅ consists of 2 to 4 amino acids each independently selected from the group consisting of alanine, serine, and valine, followed by any amino acid.

2. A composition according to claim 1, further comprising a nucleic acid segment encoding said immunogen.

3. A composition according to claim 2, wherein said immunogen is a CTL inducing-peptide.

4. A composition according to claim 2, wherein said immunogen is an antibody inducing-peptide.

5. A composition according to claim 2, wherein said immunogen is an HTL inducing-peptide.

6. A composition according to claim 2, wherein said nucleic acid segment encoding said pan DR binding oligopeptide and said nucleic acid segment encoding said immunogen are contained within one nucleic acid construct.

7. A composition according to claim 6, wherein said one nucleic acid construct is an expression vector.

8. A composition according to claim 2, wherein said nucleic acid segment encoding said pan DR binding oligopeptide and said nucleic acid segment encoding said immunogen are contained within more than one nucleic acid construct.

9. A composition according to claim 8, wherein at least one of said more than one nucleic acid constructs is an expression vector.

10. The composition of claim 2, wherein said immunogen is an antigen derived from a member of the group consisting of a pathogen, a diseased cell, a virus, a cancer cell, a fungus, a parasite and a bacterium.

11. A pharmaceutical composition comprising a pharmaceutically acceptable carrier and a composition according to claim 1.

12. A pharmaceutical composition comprising a pharmaceutically acceptable carrier and a composition according to claim 2.

13. A method of enhancing an immune response to an immunogen, said method comprising introducing a composition according to claim 1 into a subject.

14. A method of enhancing an immune response to an immunogen, said method comprising introducing a composition according to claim 2 into a subject.

15. A method according to claim 13, wherein the immune response is prophylactic.

16. A method according to claim 14, wherein the immune response is prophylactic.

17. A method according to claim 13, wherein the immune response is therapeutic.

18. A method according to claim 14,. wherein the immune response is therapeutic.
